SEQUENCE LISTING (117 sequences)

SEQ ID NO: 1 - protein, 5 aa, Artificial Sequence
  Description of Artificial Sequence Synthetic peptide
NYGMN

SEQ ID NO: 2 - protein, 17 aa, Artificial Sequence
  Description of Artificial Sequence Synthetic peptide
WINTKTGEPTYAEEFKG

SEQ ID NO: 3 - protein, 12 aa, Artificial Sequence
  Description of Artificial Sequence Synthetic peptide
GGYGSSYWYFDV

SEQ ID NO: 4 - protein, 11 aa, Artificial Sequence
  Description of Artificial Sequence Synthetic peptide
KASQDVSIAVA

SEQ ID NO: 5 - protein, 7 aa, Artificial Sequence
  Description of Artificial Sequence Synthetic peptide
SASYRYT

SEQ ID NO: 6 - protein, 9 aa, Artificial Sequence
  Description of Artificial Sequence Synthetic peptide
QQHYITPLT

SEQ ID NO: 7 - protein, 121 aa, Artificial Sequence
  Description of Artificial Sequence Synthetic polypeptide
QIQLVQSGHEVKKPGASVKVSCKASGYTFTNYGMNWVRQAPGQGLEWMGWINTKTGEPTYAEEFKGRFVFSLETSASTAYLQISSLKAEDTAMYFCGRGGYGSSYWYFDVWGQGTTVTVSS

SEQ ID NO: 8 - protein, 107 aa, Artificial Sequence
  Description of Artificial Sequence Synthetic polypeptide
DIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIK

SEQ ID NO: 9 - protein, 107 aa, Artificial Sequence
  Description of Artificial Sequence Synthetic polypeptide
DIQMTQSPSSLSASVGDRVTITCKASQDVSIAVAWYQQKPGKAPKVLIYSASYRYTGVPSRFSGSGSGTDFTFTISSLQPEDIAVYYCQQHYITPLTFGGGTKVEIK

SEQ ID NO: 10 - protein, 121 aa, Artificial Sequence
  Description of Artificial Sequence Synthetic polypeptide
QIQLVQSGHEVKKPGASVKVSCKASGYTFTNYGMNWVRQAPGQGLEWMGWINTKTGEPTYAEEFKGRFVFSLETSASTAYLQISSLKAEDMAMYFCGRGGYGSSYWYFDVWGQGTTVTVSS

SEQ ID NO: 11 - protein, 9 aa, Artificial Sequence
  Description of Artificial Sequence Synthetic peptide
LFRERYRLH

SEQ ID NO: 12 - protein, 9 aa, Artificial Sequence
  Description of Artificial Sequence Synthetic peptide
QVERTLIYY

SEQ ID NO: 13 - protein, 14 aa, Artificial Sequence
  Description of Artificial Sequence Synthetic peptide
RRLFRERYRLHPKF

SEQ ID NO: 14 - protein, 14 aa, Artificial Sequence
  Description of Artificial Sequence Synthetic peptide
RLFRERYRLHPKFV

SEQ ID NO: 15 - protein, 14 aa, Artificial Sequence
  Description of Artificial Sequence Synthetic peptide
GMAVLVITNRRKSG

SEQ ID NO: 16 - protein, 14 aa, Artificial Sequence
  Description of Artificial Sequence Synthetic peptide
MAVLVITNRRKSGK

SEQ ID NO: 17 - protein, 14 aa, Artificial Sequence
  Description of Artificial Sequence Synthetic peptide
LVAGMAVLVITNRR

SEQ ID NO: 18 - protein, 14 aa, Artificial Sequence
  Description of Artificial Sequence Synthetic peptide
AVLVITNRRKSGKY

SEQ ID NO: 19 - protein, 14 aa, Artificial Sequence
  Description of Artificial Sequence Synthetic peptide
AELRRLFRERYRLH

SEQ ID NO: 20 - protein, 14 aa, Artificial Sequence
  Description of Artificial Sequence Synthetic peptide
LFRERYRLHPKFVA

SEQ ID NO: 21 - protein, 14 aa, Artificial Sequence
  Description of Artificial Sequence Synthetic peptide
LFQGRGGLDLRVRG

SEQ ID NO: 22 - protein, 14 aa, Artificial Sequence
  Description of Artificial Sequence Synthetic peptide
VAGMAVLVITNRRK

SEQ ID NO: 23 - protein, 14 aa, Artificial Sequence
  Description of Artificial Sequence Synthetic peptide
LQVERTLIYYLDEI

SEQ ID NO: 24 - protein, 14 aa, Artificial Sequence
  Description of Artificial Sequence Synthetic peptide
ELRRLFRERYRLHP

SEQ ID NO: 25 - protein, 14 aa, Artificial Sequence
  Description of Artificial Sequence Synthetic peptide
LVRTHHILIDLRHR

SEQ ID NO: 26 - protein, 14 aa, Artificial Sequence
  Description of Artificial Sequence Synthetic peptide
AGMAVLVITNRRKS

SEQ ID NO: 27 - protein, 14 aa, Artificial Sequence
  Description of Artificial Sequence Synthetic peptide
LVITNRRKSGKYKK

SEQ ID NO: 28 - protein, 14 aa, Artificial Sequence
  Description of Artificial Sequence Synthetic peptide
DAELRRLFRERYRL

SEQ ID NO: 29 - protein, 14 aa, Artificial Sequence
  Description of Artificial Sequence Synthetic peptide
LRRLFRERYRLHPK

SEQ ID NO: 30 - protein, 14 aa, Artificial Sequence
  Description of Artificial Sequence Synthetic peptide
LDAELRRLFRERYR

SEQ ID NO: 31 - protein, 14 aa, Artificial Sequence
  Description of Artificial Sequence Synthetic peptide
VLVITNRRKSGKYK

SEQ ID NO: 32 - protein, 14 aa, Artificial Sequence
  Description of Artificial Sequence Synthetic peptide
PLQVERTLIYYLDE

SEQ ID NO: 33 - protein, 323 aa, Mus musculus [sequence omitted]

SEQ ID NO: 34 - DNA, 24 bp, Artificial Sequence
  Description of Artificial Sequence Synthetic primer
atgragwcacakwcycaggtcttt

SEQ ID NO: 35 - DNA, 25 bp, Artificial Sequence
  Description of Artificial Sequence Synthetic primer
atggagacagacacactcctgctat

SEQ ID NO: 36 - DNA, 29 bp, Artificial Sequence
  Description of Artificial Sequence Synthetic primer
atggagwcagacacactsctgytatgggt

SEQ ID NO: 37 - DNA, 32 bp, Artificial Sequence
  Description of Artificial Sequence Synthetic primer
atgaggrcccctgctcagwttyttggnwtctt

SEQ ID NO: 38 - DNA, 31 bp, Artificial Sequence
  Description of Artificial Sequence Synthetic primer
atgggcwtcaagatgragtcacakwyycwgg

SEQ ID NO: 39 - DNA, 29 bp, Artificial Sequence
  Description of Artificial Sequence Synthetic primer
atgagtgtgcycactcaggtcctggsgtt

SEQ ID NO: 40 - DNA, 31 bp, Artificial Sequence
  Description of Artificial Sequence Synthetic primer
atgtggggaycgktttyammcttttcaattg

SEQ ID NO: 41 - DNA, 28 bp, Artificial Sequence
  Description of Artificial Sequence Synthetic primer
atggaagccccagctcagcttctcttcc

SEQ ID NO: 42 - DNA, 26 bp, Artificial Sequence
  Description of Artificial Sequence Synthetic primer
atgagnmmktcnmttcanttcytggg

SEQ ID NO: 43 - DNA, 26 bp, Artificial Sequence
  Description of Artificial Sequence Synthetic primer
atgakgthcycngctcagytyctnrg

SEQ ID NO: 44 - DNA, 25 bp, Artificial Sequence
  Description of Artificial Sequence Synthetic primer
atggtrtccwcasctcagttccttg

SEQ ID NO: 45 - DNA, 27 bp, Artificial Sequence
  Description of Artificial Sequence Synthetic primer
atgtatatatgtttgttgtctatttct

SEQ ID NO: 46 - DNA, 29 bp, Artificial Sequence
  Description of Artificial Sequence Synthetic primer
atgaagttgcctgttaggctgttggtgct

SEQ ID NO: 47 - DNA, 29 bp, Artificial Sequence
  Description of Artificial Sequence Synthetic primer
atggatttwcargtgcagattwtcagctt

SEQ ID NO: 48 - DNA, 27 bp, Artificial Sequence
  Description of Artificial Sequence Synthetic primer
atggtyctyatvtccttgctgttctgg

SEQ ID NO: 49 - DNA, 27 bp, Artificial Sequence
  Description of Artificial Sequence Synthetic primer
atggtyctyatvttrctgctgctatgg

SEQ ID NO: 50 - DNA, 21 bp, Artificial Sequence
  Description of Artificial Sequence Synthetic primer
actggatggtgggaagatgga

SEQ ID NO: 51 - DNA, 25 bp, Artificial Sequence
  Description of Artificial Sequence Synthetic primer
atggcctggaytycwctywtmytct

SEQ ID NO: 52 - DNA, 23 bp, Artificial Sequence
  Description of Artificial Sequence Synthetic primer
agctcytcwgwgganggyggraa

SEQ ID NO: 53 - DNA, 25 bp, Artificial Sequence
  Description of Artificial Sequence Synthetic primer
atgrasttskggytmarctkgrttt

SEQ ID NO: 54 - DNA, 26 bp, Artificial Sequence
  Description of Artificial Sequence Synthetic primer
atgraatgsasctgggtywtyctctt

SEQ ID NO: 55 - DNA, 29 bp, Artificial Sequence
  Description of Artificial Sequence Synthetic primer
atggactccaggctcaatttagttttcct

SEQ ID NO: 56 - DNA, 26 bp, Artificial Sequence
  Description of Artificial Sequence Synthetic primer
atggctgtcytrgbgctgytcytctg

SEQ ID NO: 57 - DNA, 29 bp, Artificial Sequence
  Description of Artificial Sequence Synthetic primer
atggvttggstgtggamcttgcyattcct

SEQ ID NO: 58 - DNA, 26 bp, Artificial Sequence
  Description of Artificial Sequence Synthetic primer
atgaaatgcagctggrtyatsttctt

SEQ ID NO: 59 - DNA, 26 bp, Artificial Sequence
  Description of Artificial Sequence Synthetic primer
atggrcagrcttacwtyytcattcct

SEQ ID NO: 60 - DNA, 26 bp, Artificial Sequence
  Description of Artificial Sequence Synthetic primer
atgatggtgttaagtcttctgtacct

SEQ ID NO: 61 - DNA, 26 bp, Artificial Sequence
  Description of Artificial Sequence Synthetic primer
atgggatggagctrtatcatsytctt

SEQ ID NO: 62 - DNA, 23 bp, Artificial Sequence
  Description of Artificial Sequence Synthetic primer
atgaagwtgtggbtraactggrt

SEQ ID NO: 63 - DNA, 25 bp, Artificial Sequence
  Description of Artificial Sequence Synthetic primer
atggratggasckknrtctttmtct

SEQ ID NO: 64 - DNA, 25 bp, Artificial Sequence
  Description of Artificial Sequence Synthetic primer
atgaacttygggytsagmttgrttt

SEQ ID NO: 65 - DNA, 25 bp, Artificial Sequence
  Description of Artificial Sequence Synthetic primer
atgtacttgggactgagctgtgtat

SEQ ID NO: 66 - DNA, 23 bp, Artificial Sequence
  Description of Artificial Sequence Synthetic primer
atgagagtgctgattcttttgtg

SEQ ID NO: 67 - DNA, 28 bp, Artificial Sequence
  Description of Artificial Sequence Synthetic primer
atggattttgggctgattttttttattg

SEQ ID NO: 68 - DNA, 26 bp, Artificial Sequence
  Description of Artificial Sequence Synthetic primer
ccagggrccarkggataracngrtgg

SEQ ID NO: 69 - DNA, 363 bp, Mus musculus
  CDS (1)..(363)
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaaaactggagagccaacatatgctgaagagttcaagggacggtttgccttctctttggaaacctctgccagcactgcctatttgcagatcaacaacctcaaaaaagaggacacggctacatatttctgtggaagagggggctacggtagtagctactggtacttcgatgtctggggcgcagggaccacggtcaccgtctcctca

SEQ ID NO: 70 - protein, 121 aa, Mus musculus
QIQLVQSGPELKKPGETVKISCKASGYTFTNYGMNWVKQAPGKGLKWMGWINTKTGEPTYAEEFKGRFAFSLETSASTAYLQINNLKKEDTATYFCGRGGYGSSYWYFDVWGAGTTVTVSS

SEQ ID NO: 71 - DNA, 321 bp, Mus musculus
  CDS (1)..(321)
gacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtgggtcaggcacacagttttctctgaagatcaacagcctgcggcctgaagattttgggagttattactgtcaacatcattatggttctccgctcacgttcggtgctgggaccaagctggagctgaga

SEQ ID NO: 72 - protein, 107 aa, Mus musculus
DIQMTQSPASLSASVGETVTITCRASENIYSYLAWYQQKQGKSPQLLVYNAKTLAEGVPSRFSGSGSGTQFSLKINSLRPEDFGSYYCQHHYGSPLTFGAGTKLELR

SEQ ID NO: 73 - DNA, 39 bp, Artificial Sequence
  Description of Artificial Sequence Synthetic oligonucleotide
gatcacgcgtgtccactcccagatccagttggtgcagtc

SEQ ID NO: 74 - DNA, 33 bp, Artificial Sequence
  Description of Artificial Sequence Synthetic oligonucleotide
gtacaagcttacctgaggagacggtgaccgtgg

SEQ ID NO: 75 - DNA, 54 bp, Artificial Sequence
  Description of Artificial Sequence Synthetic oligonucleotide
gatcacgcgtgtccactcccagatccagttggtgcagtctggacctgagctgaa

SEQ ID NO: 76 - DNA, 53 bp, Artificial Sequence
  Description of Artificial Sequence Synthetic oligonucleotide
cagtctggacctgagctgaagaagcctggagcgtcagtcaaggtctcctgcaa

SEQ ID NO: 77 - DNA, 34 bp, Artificial Sequence
  Description of Artificial Sequence Synthetic oligonucleotide
taaaccctgtcctggagcctgcggcacccagttc

SEQ ID NO: 78 - DNA, 33 bp, Artificial Sequence
  Description of Artificial Sequence Synthetic oligonucleotide
caggctccaggacagggtttagagtggatgggc

SEQ ID NO: 79 - DNA, 58 bp, Artificial Sequence
  Description of Artificial Sequence Synthetic oligonucleotide
tgctgatctgcaaataggcagtgctggcagaggtttccaaagagaagacaaaccgtcc

SEQ ID NO: 80 - DNA, 57 bp, Artificial Sequence
  Description of Artificial Sequence Synthetic oligonucleotide
ctgcctatttgcagatcagcagcctcaaagcagaggacacggctatgtatttctgtg

SEQ ID NO: 81 - DNA, 50 bp, Artificial Sequence
  Description of Artificial Sequence Synthetic oligonucleotide
ctagaagcttacctgaggagacggtgaccgtggtcccttggccccagaca

SEQ ID NO: 82 - DNA, 60 bp, Artificial Sequence
  Description of Artificial Sequence Synthetic oligonucleotide
gatcacgcgtgtccactcccagatccagttggtgcagtctggacatgaggtgaagaagcc

SEQ ID NO: 83 - DNA, 22 bp, Artificial Sequence
  Description of Artificial Sequence Synthetic oligonucleotide
aatacatagccatgtcctctgc

SEQ ID NO: 84 - DNA, 22 bp, Artificial Sequence
  Description of Artificial Sequence Synthetic oligonucleotide
gcagaggacatggctatgtatt

SEQ ID NO: 85 - DNA, 31 bp, Artificial Sequence
  Description of Artificial Sequence Synthetic oligonucleotide
agaggacatggctatgtattactgtggaaga

SEQ ID NO: 86 - DNA, 22 bp, Artificial Sequence
  Description of Artificial Sequence Synthetic oligonucleotide
ctggcagaggtgtccaaagaga

SEQ ID NO: 87 - DNA, 22 bp, Artificial Sequence
  Description of Artificial Sequence Synthetic oligonucleotide
tctctttggacacctctgccag

SEQ ID NO: 88 - DNA, 34 bp, Artificial Sequence
  Description of Artificial Sequence Synthetic oligonucleotide
gatcacgcgtgtccactcccaggtccagttggtg

SEQ ID NO: 89 - DNA, 21 bp, Artificial Sequence
  Description of Artificial Sequence Synthetic oligonucleotide
gaggtgtccatagagaagaca

SEQ ID NO: 90 - DNA, 21 bp, Artificial Sequence
  Description of Artificial Sequence Synthetic oligonucleotide
tgtcttctctatggacacctc

SEQ ID NO: 91 - DNA, 22 bp, Artificial Sequence
  Description of Artificial Sequence Synthetic oligonucleotide
agccccctcttgcacagtaata

SEQ ID NO: 92 - DNA, 22 bp, Artificial Sequence
  Description of Artificial Sequence Synthetic oligonucleotide
tattactgtgcaagagggggct

SEQ ID NO: 93 - DNA, 35 bp, Artificial Sequence
  Description of Artificial Sequence Synthetic oligonucleotide
catggcgcgcgatgtgacattgtgatgacccagtc

SEQ ID NO: 94 - DNA, 63 bp, Artificial Sequence
  Description of Artificial Sequence Synthetic oligonucleotide
tgcgggatccaactgaggaagcaaagtttaaattctactcacgtttcagctccagcttggtcc

SEQ ID NO: 95 - DNA, 62 bp, Artificial Sequence
  Description of Artificial Sequence Synthetic oligonucleotide
gatcgcgcgcgatgtgacattgtgatgacccagtctccctcattcctgtccgcatcagtagg

SEQ ID NO: 96 - DNA, 43 bp, Artificial Sequence
  Description of Artificial Sequence Synthetic oligonucleotide
attcctgtccgcatcagtaggagacagggtcaccatcacctgc

SEQ ID NO: 97 - DNA, 24 bp, Artificial Sequence
  Description of Artificial Sequence Synthetic oligonucleotide
actttaggagcttttcctggtttc

SEQ ID NO: 98 - DNA, 24 bp, Artificial Sequence
  Description of Artificial Sequence Synthetic oligonucleotide
gaaaccaggaaaagctcctaaagt

SEQ ID NO: 99 - DNA, 28 bp, Artificial Sequence
  Description of Artificial Sequence Synthetic oligonucleotide
gtcttcagcctgcagagaactgatggtg

SEQ ID NO: 100 - DNA, 33 bp, Artificial Sequence
  Description of Artificial Sequence Synthetic oligonucleotide
gttctctgcaggctgaagacatcgcagtttatt

SEQ ID NO: 101 - DNA, 75 bp, Artificial Sequence
  Description of Artificial Sequence Synthetic oligonucleotide
gatcggatccaactgaggaagcaaagtttaaattctactcacgtttaatctccaccttggtcccaccaccgaacg

SEQ ID NO: 102 - DNA, 54 bp, Artificial Sequence
  Description of Artificial Sequence Synthetic oligonucleotide
gatcgcgcgcgatgtgacattgtgatgacccagtctccctcatccctgtccgca

SEQ ID NO: 103 - DNA, 24 bp, Artificial Sequence
  Description of Artificial Sequence Synthetic oligonucleotide
cgatgtcttcaggctgcagagaac

SEQ ID NO: 104 - DNA, 24 bp, Artificial Sequence
  Description of Artificial Sequence Synthetic oligonucleotide
gttctctgcagcctgaagacatcg

SEQ ID NO: 105 - DNA, 34 bp, Artificial Sequence
  Description of Artificial Sequence Synthetic oligonucleotide
gatcgcgcgcgatgtgacattcagatgacccagt

SEQ ID NO: 106 - DNA, 21 bp, Artificial Sequence
  Description of Artificial Sequence Synthetic oligonucleotide
tccactgccactgaagcgatc

SEQ ID NO: 107 - DNA, 21 bp, Artificial Sequence
  Description of Artificial Sequence Synthetic oligonucleotide
gatcgcttcagtggcagtgga

SEQ ID NO: 108 - DNA, 23 bp, Artificial Sequence
  Description of Artificial Sequence Synthetic oligonucleotide
cactgaagcgactagggactcca

SEQ ID NO: 109 - DNA, 23 bp, Artificial Sequence
  Description of Artificial Sequence Synthetic oligonucleotide
tggagtccctagtcgcttcagtg

SEQ ID NO: 110 - DNA, 35 bp, Artificial Sequence
  Description of Artificial Sequence Synthetic oligonucleotide
tctgcagcctgaagacatcgcaacttattactgtc

SEQ ID NO: 111 - protein, 121 aa, Artificial Sequence
  Description of Artificial Sequence Synthetic polypeptide
QIQLVQSGHEVKKPGASVKVSCKASGYTFTNYGMNWVRQAPGQGLEWMGWINTKTGEPTYAEEFKGRFVFSMDTSASTAYLQISSLKAEDMAMYYCGRGGYGSSYWYFDVWGQGTTVTVSS

SEQ ID NO: 112 - protein, 121 aa, Artificial Sequence
  Description of Artificial Sequence Synthetic polypeptide
QIQLVQSGHEVKKPGASVKVSCKASGYTFTNYGMNWVRQAPGQGLEWMGWINTKTGEPTYAEEFKGRFVFSLDTSASTAYLQISSLKAEDMAMYYCGRGGYGSSYWYFDVWGQGTTVTVSS

SEQ ID NO: 113 - protein, 121 aa, Artificial Sequence
  Description of Artificial Sequence Synthetic polypeptide
QIQLVQSGHEVKKPGASVKVSCKASGYTFTNYGMNWVRQAPGQGLEWMGWINTKTGEPTYAEEFKGRFVFSLETSASTAYLQISSLKAEDMAMYYCGRGGYGSSYWYFDVWGQGTTVTVSS

SEQ ID NO: 114 - protein, 121 aa, Artificial Sequence
  Description of Artificial Sequence Synthetic polypeptide
QIQLVQSGPELKKPGASVKVSCKASGYTFTNYGMNWVRQAPGQGLEWMGWINTKTGEPTYAEEFKGRFVFSLETSASTAYLQISSLKAEDMAMYFCGRGGYGSSYWYFDVWGQGTTVTVSS

SEQ ID NO: 115 - protein, 107 aa, Artificial Sequence
  Description of Artificial Sequence Synthetic polypeptide
DIQMTQSPSSLSASVGDRVTITCKASQDVSIAVAWYQQKPGKAPKVLIYSASYRYTGVPSRFSGSGSGTDFTFTISSLQPEDIATYYCQQHYITPLTFGGGTKVEIK

SEQ ID NO: 116 - protein, 107 aa, Artificial Sequence
  Description of Artificial Sequence Synthetic polypeptide
DIVMTQSPSSLSASVGDRVTITCKASQDVSIAVAWYQQKPGQAPKVLIYSASYRYTGVPDRFTGSGSGTDFTFTISSLQPEDIAVYYCQQHYITPLTFGGGTKVEIK

SEQ ID NO: 117 - protein, 107 aa, Artificial Sequence
  Description of Artificial Sequence Synthetic polypeptide
DIVMTQSPSFLSASVGDRVTITCKASQDVSIAVAWYQQKPGKAPKVLIYSASYRYTGVPDRFTGSGSGTDFTFTISSLQAEDIAVYYCQQHYITPLTFGGGTKVEIK

(19) United States
(12) Patent Application Publication
(10) Pub. No.: US 2008/0131428 A1
(43) Pub. Date: Jun. 5, 2008

(54) Cytotoxicity mediation of cells evidencing surface expression of TROP-2

(75) Inventors: David S. F. Young, Toronto (CA); Helen P. Findlay, Toronto (CA); Susan E. Hahn, Toronto (CA); Luis A. G. DaCruz, Toronto (CA); Alison L. Ferry, Thornhill (CA)

Correspondence Address: MCHALE & SLAVIN, P.A., 2855 PGA BLVD, PALM BEACH GARDENS, FL, 33410, US

(73) Assignee: Arius Research, Inc.

(21) Appl. No.: 11/807,837

(22) Filed: May 30, 2007

Related U.S. Application Data
(63) Continuation-in-part of application No. 11/709,676, filed on Feb. 22, 2007.
(60) Provisional application No. 60/776,466, filed on Feb. 24, 2006.

(51) Int. Cl. A61K 39/00; A61K 39/395; G01N 33/53; C12N 5/04; C07K 16/00

(52) U.S. Cl. 424/133.1; 424/141.1; 435/7.23; 435/375; 530/387.7; 530/387.3

(57) ABSTRACT

This invention relates to the staging, diagnosis and treatment of cancerous diseases (both primary tumors and tumor metastases), particularly to the mediation of cytotoxicity of tumor cells; and most particularly to the use of cancerous disease modifying antibodies (CDMAB), optionally in combination with one or more CDMAB/chemotherapeutic agents, as a means for initiating the cytotoxic response. The invention further relates to binding assays, which utilize the CDMAB of the instant invention. The anti-cancer antibodies can be conjugated to toxins, enzymes, radioactive compounds, cytokines, interferons, target or reporter moieties and hematogenous cells.

### REFERENCE TO RELATED APPLICATIONS

This application is a continuation-in-part to U.S. patent application Ser. No. 11/709,676, filed on Feb. 22, 2007, which claims benefit of the filing date of Provisional Application 60/776,466, filed on Feb. 24, 2006, the contents of which are herein incorporated by reference.

### FIELD OF THE INVENTION

This invention relates to the diagnosis and treatment of cancerous diseases, particularly to the mediation of cytotoxicity of tumor cells; and most particularly to the use of cancerous disease modifying antibodies (CDMAB), optionally in combination with one or more CDMAB/chemotherapeutic agents, as a means for initiating the cytotoxic response. The invention further relates to binding assays, which utilize the CDMAB of the instant invention.

### BACKGROUND OF THE INVENTION

TROP-2 is a cell surface glycoprotein expressed on most carcinomas, as well as some normal human tissues. It was initially defined as a molecule recognized by two murine monoclonal antibodies raised to a human choriocarcinoma cell line BeWo that recognized an antigen on human trophoblast cells (Faulk 1978). The same molecule was independently discovered by other investigators which led to multiple names describing the same antigen. Hence, TROP-2 was also referred to as GA733-1 and epithelial glycoprotein-1 (EGP-1) (Basu 1995, Fomaro 1995).

The TROP-2 gene is an intronless gene that was thought to have been formed through the retroposition of a homologous gene GA 733-2 (also known as epithelial glycoprotein-2, EpCAM and Trop-1) via an RNA intermediate. The TROP-2 gene has been mapped to chromosome 1p32 (Calabrese 2001). The protein component of TROP-2 has a molecular mass of approximately 35 kilodaltons. Its mass may be increased by 11-13 kilodaltons with heterogeneous N-linked glycosylation of its extracellular domain. There are many cysteine residues in the extracellular domain which could form disulfide bridge sites. TROP-2 is a substrate for protein kinase C, a Ca²⁺ dependent protein kinase and the intracellular serine 303 residue has been shown to be phosphorylated (Basu 1995). It has also been shown that crossing-linking of TROP-2 with anti-TROP-2 antibodies transduced a calcium signal as shown by a rise in cytoplasmic Ca²⁺ (Ripani 1998). These data support signal transduction as a physiological function of TROP-2, although to date no physiological ligand has been identified. An association between TROP-2 expression and cancer has been shown in a report in which TROP-2 was identified as a member of a group of genes reported to be the most highly overexpressed in ovarian serous papillary carcinoma compared to normal ovarian epithelium in a large-scale gene expression analysis using cDNA microarray technology (Santin 2004). A recent study, analyzing 74 colorectal human cancer samples by quantitative real-time RT-PCR and 34 of the samples by immunohistochemistry, examined TROP-2 expression levels in cancer and normal patient sections. TROP-2 was found to be more highly expressed in cancer versus normal patient samples, and the study further demonstrated a correlation between TROP-2 expression levels and biological aggressiveness. High levels of TROP-2 were found to be associated with poor prognosis, a decrease in patient survival and an increase in the frequency of liver metastases (Ohmachi 2006), suggesting that TROP-2 may be useful as a prognostic indicator and may be an attractive therapeutic target.

The expression profile of TROP-2 has been elucidated through immunohistochemistry (IHC) and flow cytometry studies using many different TROP-2 antibodies. Anti-TROP-2 antibodies 162-25.3 and 162-46.2 were produced through immunization of mice with the human choriocarcinoma cell line BeWo, and were investigated for their reactivity to a series of tumor and lymphoid cell lines and peripheral blood mononuclear cells. In this study both antibodies appeared to be trophoblast specific, staining 3 of the 4 choriocarcinoma cell lines tested, while none of the other lymphoid or tumor cell lines (representing fibrosarcoma, cervical sarcoma, colon carcinoma, melanoma, neuroblastoma, erythroleukemia) were stained in an indirect immunofluorescence FACS assay. In addition, none of the normal peripheral blood cells were stained. The antibodies were tested for staining of formalin-fixed paraffin-embedded placenta tissue sections and frozen normal sections of liver, kidney, spleen, thymus and lymph node tissues. The placenta tissue sections were stained with both antibodies, while there was no staining of the other normal tissues (Lipinski 1981). These two antibodies have strictly been reported for use in in vitro diagnostic studies.

Anti-TROP-2 antibody MOv16 was generated through the immunization of mice with a crude membrane preparation of poorly differentiated ovarian carcinoma OvCa4343/83. MOv16 was tested for reactivity to a series of frozen tissue sections of benign and malignant ovarian tumors. MOv16 reacted with 31 of 54 malignant ovarian tumors and 2 of 16 benign ovarian tumors. Of the 5 mucinous ovarian tumors that were tested, MOv16 was completely unreactive. MOv16 was also tested for reactivity to frozen sections of non-ovarian malignant tumors where it was found to bind 117 of 189 breast carcinoma sections and 12 of 18 lung carcinoma sections. MOv16 was completely unreactive on 16 non-epithelial tumors that were tested (including liposarcomas, chondrosarcomas, endotheliomas, histiocytomas and dysgerminomas). When tested on frozen normal tissue sections, MOv-16 was reactive with breast, pancreas, kidney and prostate sections. MOv 16 reactivity was reported to be negative on lung, spleen, skin, ovary, thyroid, parotid gland, stomach, larynx, uterus and colon sections, though the number of tissue sections that were used was not reported. The authors noted that frozen tissue sections were used because MOv16 was unreactive to paraffin embedded tissues (Miotti 1987). This antibody has also only been reported for use in in vitro diagnositic studies.

Anti-TROP-2 antibody Rs7-3G11 (RS7) was generated through the immunization of mice with a crude membrane preparation derived from a surgically removed human primary squamous cell carcinoma of the lung. IHC was used to examine the staining of RS7 on frozen sections of human tumor and normal tissues. RS7 bound to 33 of the 40 sections representing tumors of the breast, colon, kidney, lung, prostate and squamous cell cancer. Of the normal tissues RS7 bound to 16 of 20 sections of breast, colon, kidney, liver, lung and prostate tissues while none of the five sections of spleen tissue were stained. In this study the authors noted that it appeared that antigen density in tumors was higher than in normal epithelial tissues (Stein 1990).

Additional studies of the tissue specificity of RS7 were carried out on both tumor and normal tissues. RS7 was tested on a panel of frozen tumor sections and bound to 65 of the 77 sections representing tumors of the lung, stomach, kidney, bladder, colon, breast, ovary, uterus and prostate. There was no binding to the 5 lymphomas tested. RS7 was tested on a panel of 85 frozen human normal tissue sections composed of a total of 24 tissue types. 39 sections of 13 normal tissues (lung, bronchus, trachea, esophagus, colon, liver, pancreas, kidney, bladder, skin, thyroid, breast and prostate) were stained by RS7. The authors of this study noted that in the tissues in which positive staining was observed, the reactivity was generally restricted to epithelial cells, primarily in ducts or glands. It was also noted that this study was limited to frozen sections since it was observed that RS7 was not reactive on formalin-fixed paraffin-embedded sections (Stein 1993).

Polyclonal anti-TROP-2 antibodies were prepared by immunizing mice with a synthetic peptide corresponding to amino acid positions between 169 and 182 of the cytoplasmic domain of human TROP-2. The polyclonal antibodies were tested on a tissue array slide that contained formalin-fixed human esophageal hyperplasia and carcinoma tissues. Ten of the 55 carcinoma specimens displayed heavy staining with the polyclonal antibodies, while the mild hyperplasia tissue stained very weakly, indicating expression levels may be related to malignant transformation (Nakashima 2004).

Overall, IHC reactivity patterns obtained with different anti-TROP-2 antibodies were consistent. Expression in cancer was seen primarily in carcinomas, and most carcinomas were reactive. In normal tissues, expression appeared to be limited to cells of epithelial origin, and there was some evidence that staining of carcinomas was stronger than staining of corresponding normal epithelial tissues.

In addition to being used in IHC studies, antibody RS7 was tested in in vivo models with initial experiments consisting of tumor targeting studies in nude mouse xenograft models. Radiolabeled RS7 injected i.v. was shown to accumulate specifically in the tumor of mice bearing either Calu-3 (lung adenocarcinoma) or GW-39 (colon carcinoma) tumors (Stein 1990). Further studies were done to investigate the biodistribution of radiolabeled RS7 in a xenograft system and to study the therapeutic potential of RS7 as an immunoconjugate. In this study the therapeutic efficacy of ¹³¹I-labeled RS7 F(ab′)₂ was investigated in nude mice bearing Calu-3 human lung adenocarcinoma xenografts. Three weeks following inoculation of the mice with Calu-3 cells, when the tumors had reached a size of approximately 0.3-0.9 grams, groups of 6-7 mice were treated with a single dose i.v. of either 1.0 mCi ¹³¹I-RS7-F(ab′)₂ or 1.5 mCi ¹³¹I-RS7-F(ab′)₂ and compared to a similar group of untreated control mice. The single dose of 1.0 mCi ¹³¹I-RS7-F(ab′)₂ resulted in tumor growth suppression for approximately 5 weeks, while the single dose of 1.5 mCi ¹³¹I-RS7-F(ab′)₂ resulted in tumor regression, and the mean tumor size did not exceed the pre-therapy size until the eighth week after radioantibody injection. Mice receiving the 1.5 mCi ¹³¹I-RS7-F(ab′)₂ dose experienced a mean body weight loss of 18.7 percent, indicating there was toxicity associated with the treatment. In this study, effects of treatment with naked RS7 or the F(ab′)₂ fragment of RS7 were not tested (Stein 1994a). Another study was done to test the efficacy of ¹³¹I-RS7 in a MDA-MB-468 breast cancer xenograft model. Groups of ten mice bearing MDA-MB-468 tumors of approximately 0.1 cm³ were treated with a single dose i.v. of either 250 microcuries ¹³¹I-RS7 or 250 microcuries ¹³¹I-Ag8 (an isotype matched control antibody). Groups of six mice were treated with a single dose i.v. of 30 micrograms of either unlabeled RS7 or Ag8. Complete regression of the tumors (except for one animal that had a transient reappearance of tumor) was seen in the animals treated with ¹³¹I-RS7, which lasted for the duration of the 11 week observation period. Tumor regression was also seen in ¹³¹I-Ag8 treated mice, though was only observed between 2 weeks and 5 weeks with tumors either persisting or continuing to grow for the remainder of the study. Tumor growth of mice that received unlabeled RS7 or Ag8 was not inhibited and there did not appear to be any differences in the mean tumor volume of RS7 treated mice compared to the Ag8 treated mice. Two additional groups of 10 mice bearing larger MDA-MB-468 tumors of approximately 0.2-0.3 cm³ were treated with a slightly higher single dose of either 275 microcuries ¹³¹I-Rs7 or 275 microcuries ¹³¹Ag8 and compared to a similar group of untreated mice. Tumor volume was measured weekly for 15 weeks. Although in this case there was a significant difference in tumor growth between the ¹³¹I-RS7 treated mice compared to the untreated mice, there was no significant difference in the tumor growth of the ¹³¹I-RS7 compared to the ¹³¹I-Ag8 treated mice, indicating a portion of the efficacy may have been due to non-specific effects of the radiation. Unlabeled antibodies were not tested in mice containing 0.2-0.3 cm³ tumors (Shih 1995).

There have been numerous additional studies examining the efficacy of RS7 as an immunoconjugate with an attempt to select the optimal radiolabel for radioimmunotherapy (Stein 2001a, Stein 2001b, Stein 2003). A humanized version of RS7 has also been generated; however it has only been tested in preclinical xenograft models as a radioconjugate (Govindan 2004). These studies show similar positive effects as the previously described studies with RS7, however in one study, even when radiolabeled RS7 was delivered at a previously determined maximum tolerable dose, toxicity occurred leading to death in some mice (Stein 2001a). Although effective treatment of xenograft tumors in mice was achieved with radiolabeled RS7 in these studies, naked RS7 was not evaluated.

Immunizing mice with neuraminidase pre-treated H3922 human breast carcinoma cells produced the anti-TROP-2 monoclonal antibody BR110 (as disclosed in U.S. Pat. No. 5,840,854, refer to Prior patents section). By immunohistology, using human frozen tissue specimens, BR10 was shown to react with a wide range of human carcinoma specimens including those of the lung, colon, breast, ovarian, kidney, esophagus, pancreas, skin, lung and tonsil. No human normal tissue sections were tested. In vitro studies demonstrated that BR110 had no ADCC or CDC activity on the human carcinoma cell lines H3396 or H3922. In vitro studies analyzing the cytotoxicity of BR110-immunotoxins was performed on the human cancer cell lines H3619, H2987, MCF-7, H3396 and H2981. The EC₅₀ for the cell lines tested was 0.06, 0.001, 0.05, 0.09 and >5 micrograms/mL respectively. No cytotoxicity data was disclosed for the naked BR110 antibody. No in vivo data was disclosed for the naked or immunoconjugated BR110.

A number of additional antibodies have been generated that target TROP-2, such as MR54, MR6 and MR23 which were generated from immunization of mice with the ovarian cancer cell line Colo 316 (Stein 1994b) and antibody T16 which was generated by immunization of mice with the T24 bladder cancer cell line (Fradet 1984). The use of these antibodies has been limited to biochemical characterization of the TROP-2 antigen and cell line and tissue expression studies. There have been no reports of anti-cancer efficacy of these antibodies, either in vitro or in vivo. RS7 was the only antibody that was tested for therapeutic efficacy in preclinical cancer models, with its use being limited to a carrier of radioisotope. There are no reports of any naked TROP-2 antibodies exhibiting therapeutic efficacy in clinical studies or in preclinical cancer models either in vitro or in vivo.

Monoclonal Antibodies as Cancer Therapy: Each individual who presents with cancer is unique and has a cancer that is as different from other cancers as that person's identity. Despite this, current therapy treats all patients with the same type of cancer, at the same stage, in the same way. At least 30 percent of these patients will fail the first line therapy, thus leading to further rounds of treatment and the increased probability of treatment failure, metastases, and ultimately, death. A superior approach to treatment would be the customization of therapy for the particular individual. The only current therapy which lends itself to customization is surgery. Chemotherapy and radiation treatment cannot be tailored to the patient, and surgery by itself, in most cases is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens; thus giving rise to the belief that monoclonal antibodies can serve as “Magic Bullets” to eliminate cancer cells. However, it is now widely recognized that no single monoclonal antibody can serve in all instances of cancer, and that monoclonal antibodies can be deployed, as a class, as targeted cancer treatments. Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remission or responses. Furthermore, there was a lack of reproducibility and there was no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least four clinical trials for human breast cancer which produced only one responder from at least 47 patients using antibodies against specific antigens or based on tissue selectivity. It was not until 1998 that there was a successful clinical trial using a humanized anti-Her2/neu antibody (Herceptin®) in combination with CISPLATIN. In this trial 37 patients were assessed for responses of which about a quarter had a partial response rate and an additional quarter had minor or stable disease progression. The median time to progression among the responders was 8.4 months with median response duration of 5.3 months.

Herceptin® was approved in 1998 for first line use in combination with Taxol®. Clinical study results showed an increase in the median time to disease progression for those who received antibody therapy plus Taxol® (6.9 months) in comparison to the group that received Taxol® alone (3.0 months). There was also a slight increase in median survival; 22 versus 18 months for the Herceptin® plus Taxol® treatment arm versus the Taxol® treatment alone arm. In addition, there was an increase in the number of both complete (8 versus 2 percent) and partial responders (34 versus 15 percent) in the antibody plus Taxol® combination group in comparison to Taxol® alone. However, treatment with Herceptin® and Taxol® led to a higher incidence of cardiotoxicity in comparison to Taxol® treatment alone (13 versus 1 percent respectively). Also, Herceptin® therapy was only effective for patients who over express (as determined through immunohistochemistry (IHC) analysis) the human epidermal growth factor receptor 2 (Her2/neu), a receptor, which currently has no known function or biologically important ligand; approximately 25 percent of patients who have metastatic breast cancer. Therefore, there is still a large unmet need for patients with breast cancer. Even those who can benefit from Herceptin® treatment would still require chemotherapy and consequently would still have to deal with, at least to some degree, the side effects of this kind of treatment.

The clinical trials investigating colorectal cancer involve antibodies against both glycoprotein and glycolipid targets. Antibodies such as 17-1A, which has some specificity for adenocarcinomas, has undergone Phase 2 clinical trials in over 60 patients with only 1 patient having a partial response. In other trials, use of 17-1A produced only 1 complete response and 2 minor responses among 52 patients in protocols using additional cyclophosphamide. To date, Phase III clinical trials of 17-1A have not demonstrated improved efficacy as adjuvant therapy for stage III colon cancer. The use of a humanized murine monoclonal antibody initially approved for imaging also did not produce tumor regression.

Only recently have there been any positive results from colorectal cancer clinical studies with the use of monoclonal antibodies. In 2004, ERBITUX® was approved for the second line treatment of patients with EGFR-expressing metastatic colorectal cancer who are refractory to irinotecan-based chemotherapy. Results from both a two-arm Phase II clinical study and a single arm study showed that ERBITUX® in combination with irinotecan had a response rate of 23 and 15 percent respectively with a median time to disease progression of 4.1 and 6.5 months respectively. Results from the same two-arm Phase II clinical study and another single arm study showed that treatment with ERBITUX® alone resulted in an 11 and 9 percent response rate respectively with a median time to disease progression of 1.5 and 4.2 months respectively.

Consequently in both Switzerland and the United States, ERBITUX® treatment in combination with irinotecan, and in the United States, ERBITUX® treatment alone, has been approved as a second line treatment of colon cancer patients who have failed first line irinotecan therapy. Therefore, like Herceptin®, treatment in Switzerland is only approved as a combination of monoclonal antibody and chemotherapy. In addition, treatment in both Switzerland and the US is only approved for patients as a second line therapy. Also, in 2004, AVASTIN® was approved for use in combination with intravenous 5-fluorouracil-based chemotherapy as a first line treatment of metastatic colorectal cancer. Phase III clinical study results demonstrated a prolongation in the median survival of patients treated with AVASTIN® plus 5-fluorouracil compared to patients treated with 5-fluourouracil alone (20 months versus 16 months respectively). However, again like Herceptin® and ERBITUX®, treatment is only approved as a combination of monoclonal antibody and chemotherapy.

There also continues to be poor results for lung, brain, ovarian, pancreatic, prostate, and stomach cancer. The most promising recent results for non-small cell lung cancer came from a Phase II clinical trial where treatment involved a monoclonal antibody (SGN-15; dox-BR96, anti-Sialyl-LeX) conjugated to the cell-killing drug doxorubicin in combination with the chemotherapeutic agent TAXOTERE®. TAXOTERE® is the only FDA approved chemotherapy for the second line treatment of lung cancer. Initial data indicate an improved overall survival compared to TAXOTERE® alone. Out of the 62 patients who were recruited for the study, two-thirds received SGN-15 in combination with TAXOTERE® while the remaining one-third received TAXOTERE® alone. For the patients receiving SGN-15 in combination with TAXOTERE®, median overall survival was 7.3 months in comparison to 5.9 months for patients receiving TAXOTERE® alone. Overall survival at 1 year and 18 months was 29 and 18 percent respectively for patients receiving SGN-15 plus TAXOTERE® compared to 24 and 8 percent respectively for patients receiving TAXOTERE® alone. Further clinical trials are planned.

Preclinically, there has been some limited success in the use of monoclonal antibodies for melanoma. Very few of these antibodies have reached clinical trials and to date none have been approved or demonstrated favorable results in Phase III clinical trials.

The discovery of new drugs to treat disease is hindered by the lack of identification of relevant targets among the products of 30,000 known genes that could contribute to disease pathogenesis. In oncology research, potential drug targets are often selected simply due to the fact that they are over-expressed in tumor cells. Targets thus identified are then screened for interaction with a multitude of compounds. In the case of potential antibody therapies, these candidate compounds are usually derived from traditional methods of monoclonal antibody generation according to the fundamental principles laid down by Kohler and Milstein (1975, Nature, 256, 495-497, Kohler and Milstein). Spleen cells are collected from mice immunized with antigen (e.g. whole cells, cell fractions, purified antigen) and fused with immortalized hybridoma partners. The resulting hybridomas are screened and selected for secretion of antibodies which bind most avidly to the target. Many therapeutic and diagnostic antibodies directed against cancer cells, including Herceptin® and RITUXIMAB, have been produced using these methods and selected on the basis of their affinity. The flaws in this strategy are two-fold. Firstly, the choice of appropriate targets for therapeutic or diagnostic antibody binding is limited by the paucity of knowledge surrounding tissue specific carcinogenic processes and the resulting simplistic methods, such as selection by overexpression, by which these targets are identified. Secondly, the assumption that the drug molecule that binds to the receptor with the greatest affinity usually has the highest probability for initiating or inhibiting a signal may not always be the case.

Despite some progress with the treatment of breast and colon cancer, the identification and development of efficacious antibody therapies, either as single agents or co-treatments, have been inadequate for all types of cancer.

### Prior Patents:

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas and not dependent upon the epithelial tissue of origin.

U.S. Pat. No. 5,783,186 is drawn to Anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single-chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an antinuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal antinuclear autoantibody from an aged mammal, and a hybridoma cell line producing a monoclonal antinuclear autoantibody.

U.S. Pat. No. 5,840,854 discloses a specific antibody, BR110 directed against GA733-1. This patent discloses in vitro function for BR110 as an immunotoxin conjugate. There was no in vitro function as a naked antibody disclosed for this antibody. There was also no in vivo function disclosed for this antibody.

U.S. Pat. No. 6,653,104 claims immunotoxin-conjugated antibodies, including but not limited to RS7, directed against a host of antigens, including but not limited to EGP-1. The immunotoxin is limited to those possessing ribonucleolytic activity. However, the examples disclose only a specific immunotoxin-conjugated antibody, LL2, directed against CD22. There was no in vitro or in vivo function for RS7 disclosed in this application.

U.S. Application No. 20040001825A1 discloses a specific antibody, RS7 directed against EGP-1. This application discloses in vitro function for RS7 as a radiolabeled conjugate. There was no in vitro function as a naked antibody disclosed for this antibody. This application also discloses in vivo function for RS7 resulting from radiolabled and unlabeled conjugate administered sequentially. However, this study was limited to one patient and it is unknown whether any of the observed function was due to the unlabeled antibody. There was no in vivo function for RS7 resulting from the administration of the naked antibody.

### SUMMARY OF THE INVENTION

This application utilizes methodology for producing patient specific anti-cancer antibodies taught in the U.S. Pat. No. 6,180,357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. These antibodies can also be used for the prevention of cancer by way of prophylactic treatment. Unlike antibodies generated according to traditional drug discovery paradigms, antibodies generated in this way may target molecules and pathways not previously shown to be integral to the growth and/or survival of malignant tissue. Furthermore, the binding affinities of these antibodies are suited to requirements for initiation of the cytotoxic events that may not be amenable to stronger affinity interactions. Also, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties, enzymes e.g. biotin conjugated enzymes, cytokines, interferons, target or reporter moieties or hematogenous cells, thereby forming an antibody conjugate. The CDMAB can be used alone or in combination with one or more CDMAB/chemotherapeutic agents.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies, and a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

In addition to anti-cancer antibodies, the patient can elect to receive the currently recommended therapies as part of a multi-modal regimen of treatment. The fact that the antibodies isolated via the present methodology are relatively non-toxic to non-cancerous cells allows for combinations of antibodies at high doses to be used, either alone, or in conjunction with conventional therapy. The high therapeutic index will also permit re-treatment on a short time scale that should decrease the likelihood of emergence of treatment resistant cells.

If the patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and an anti-cancer antibody conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody dependent cellular cytotoxicity (ADCC) or complement dependent cytotoxicity (CDC). For example murine IgM and IgG2a antibodies can activate human complement by binding the C-1 component of the complement system thereby activating the classical pathway of complement activation which can lead to tumor lysis. For human antibodies the most effective complement activating antibodies are generally IgM and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

The cytotoxicity mediated through the Fc region requires the presence of effector cells, their corresponding receptors, or proteins e.g. NK cells, T-cells and complement. In the absence of these effector mechanisms, the Fc portion of an antibody is inert. The Fc portion of an antibody may confer properties that affect the pharmacokinetics of an antibody in vivo, but in vitro this is not operative.

Another possible mechanism of antibody mediated cancer killing may be through the use of antibodies that function to catalyze the hydrolysis of various chemical bonds in the cell membrane and its associated glycoproteins or glycolipids, so-called catalytic antibodies.

There are three additional mechanisms of antibody-mediated cancer cell killing. The first is the use of antibodies as a vaccine to induce the body to produce an immune response against the putative antigen that resides on the cancer cell. The second is the use of antibodies to target growth receptors and interfere with their function or to down regulate that receptor so that its function is effectively lost. The third is the effect of such antibodies on direct ligation of cell surface moieties that may lead to direct cell death, such as ligation of death receptors such as TRAIL R1 or TRAIL R2, or integrin molecules such as alpha V beta 3 and the like.

The clinical utility of a cancer drug is based on the benefit of the drug under an acceptable risk profile to the patient. In cancer therapy survival has generally been the most sought after benefit, however there are a number of other well-recognized benefits in addition to prolonging life. These other benefits, where treatment does not adversely affect survival, include symptom palliation, protection against adverse events, prolongation in time to recurrence or disease-free survival, and prolongation in time to progression. These criteria are generally accepted and regulatory bodies such as the U.S. Food and Drug Administration (F.D.A.) approve drugs that produce these benefits (Hirschfeld et al. Critical Reviews in Oncology/Hematolgy 42:137-143 2002). In addition to these criteria it is well recognized that there are other endpoints that may presage these types of benefits. In part, the accelerated approval process granted by the U.S. F.D.A. acknowledges that there are surrogates that will likely predict patient benefit. As of year-end 2003, there have been sixteen drugs approved under this process, and of these, four have gone on to full approval, i.e., follow-up studies have demonstrated direct patient benefit as predicted by surrogate endpoints. One important endpoint for determining drug effects in solid tumors is the assessment of tumor burden by measuring response to treatment (Therasse et al. Journal of the National Cancer Institute 92(3):205-216 2000). The clinical criteria (RECIST criteria) for such evaluation have been promulgated by Response Evaluation Criteria in Solid Tumors Working Group, a group of international experts in cancer. Drugs with a demonstrated effect on tumor burden, as shown by objective responses according to RECIST criteria, in comparison to the appropriate control group tend to, ultimately, produce direct patient benefit. In the pre-clinical setting tumor burden is generally more straightforward to assess and document. In that pre-clinical studies can be translated to the clinical setting, drugs that produce prolonged survival in pre-clinical models have the greatest anticipated clinical utility. Analogous to producing positive responses to clinical treatment, drugs that reduce tumor burden in the pre-clinical setting may also have significant direct impact on the disease. Although prolongation of survival is the most sought after clinical outcome from cancer drug treatment, there are other benefits that have clinical utility and it is clear that tumor burden reduction, which may correlate to a delay in disease progression, extended survival or both, can also lead to direct benefits and have clinical impact (Eckhardt et al. Developmental Therapeutics: Successes and Failures of Clinical Trial Designs of Targeted Compounds; ASCO Educational Book, 39^{th} Annual Meeting, 2003, pages 209-219).

Using substantially the process of U.S. Pat. No. 6,180,357, and as disclosed in U.S. patent application Ser. No. 11/709,676 the contents of each of which are herein incorporated by reference, the mouse monoclonal antibody, AR47A6.4.2 was obtained following immunization of mice with cells from human ovarian tumor tissue. The AR47A6.4.2 antigen was expressed on the cell surface of a wide range of human cell lines from different tissue origins. The ovarian cancer cell line OVCAR-3 was susceptible to the cytotoxic effect of AR47A6.4.2 in vitro.

The result of AR47A6.4.2 cytotoxicity against human cancer cells in vitro was further extended by demonstrating its anti-tumor activity in vivo (as disclosed in Ser. No. 11/709,676). AR47A6.4.2 prevented tumor growth and reduced tumor burden in an in vivo prophylactic BxPC-3 model of human pancreatic cancer. On day 49 post-implantation, the last day of treatment, the mean tumor volume in the AR47A6.4.2 treated group was 53 percent less than that of the buffer control-treated group (p<0.05). There were no clinical signs of toxicity throughout the study. In summary, AR47A6.4.2 was well-tolerated and decreased the tumor burden in this human pancreatic cancer xenograft model.

To further determine the efficacy of AR47A6.4.2 on the BxPC-3 model of human pancreatic cancer, the antibody was tested on an established BxPC-3 xenograft model (as disclosed in Ser. No. 11/709,676). AR47A6.4.2 significantly reduced tumor burden in an established model of human pancreatic cancer. On day 54, one day after the last dose of antibody was administered, AR47A6.4.2-treated animals had a mean tumor volume that was 40 percent of the mean tumor volume in the control-treated animals (p<0.0001). These results correspond to a mean T/C of 30 percent for AR47A6.4.2. There were no clinical signs of toxicity throughout the study. In summary, AR47A6.4.2 was well-tolerated and decreased the tumor burden in this established human pancreatic cancer xenograft model. AR47A6.4.2 has demonstrated efficacy in both a preventative and established model of human pancreatic cancer.

AR47A6.4.2 has demonstrated anti-cancer effects in against a human pancreatic cancer model. To extend this finding AR47A6.4.2 was tested on a xenograft model of PL45 human pancreatic cancer (as disclosed in Ser. No. 11/79,676). AR47A6.4.2 completely inhibited tumor growth in the PL45 in vivo prophylactic model of human pancreatic cancer. Treatment with ARIUS antibody AR47A6.4.2 reduced the growth of PL45 tumors by nearly 100 percent (p=0.0005, t-test), compared to the buffer-treated group, as determined on day 77, 20 days after the last dose of antibody when almost all mice in control and antibody-treated group were living. At day 102, 45 days after last dose, all mice in the control group had been removed from the study due to tumor volume. However AR47A6.4.2 still demonstrated almost complete inhibition of tumor growth and 4 mice (some mice had been lost due to non-cancer related incidents) in that group were still alive. There were no obvious clinical signs of toxicity throughout the study. In summary, AR47A6.4.2 was well-tolerated and almost completely inhibited the tumor growth in this human pancreatic cancer xenograft model. AR47A6.4.2 treatment also demonstrated increased survival in comparison to buffer treatment. AR47A6.4.2 therefore has demonstrated efficacy in two different models of human pancreatic cancer.

AR47A6.4.2 has demonstrated anti-cancer properties against two different human pancreatic cancer xenograft models. To determine the efficacy of AR47A6.4.2 against a different human cancer xenograft model, the antibody was tested on a PC-3 prostate cancer xenograft model (as disclosed in Ser. No. 11/709,676). AR47A6.4.2 inhibited tumor growth in the PC-3 in vivo prophylactic model of human prostate adenocarcinoma cells. Treatment with ARIUS antibody AR47A6.4.2 reduced the growth of PC-3 tumors by 60.9 percent (p=0.00037, t-test), compared to the buffer treated group, as determined on day 32 after 5 doses of treatment with antibody when almost all mice in control and antibody-treated group were still alive. All mice in the control group had been removed from the study by day 47, 3 days before the last dose of antibody, due to tumor volume/lesions. At day 77, 27 days after last dose of antibody, 40 percent of the mice in the AR47A6.4.2-treated group still were still alive. There were no obvious clinical signs of toxicity throughout the study. In summary, AR47A6.4.2 was well-tolerated and significantly inhibited the tumor growth in this human prostate cancer xenograft model. Treatment with antibody also demonstrated a survival benefit in comparison to the control group. AR47A6.4.2 has demonstrated efficacy against two different human cancer indications: pancreatic and prostate.

AR47A6.4.2 has demonstrated anti-cancer properties against two different human pancreatic and a prostate cancer xenograft model. To determine the efficacy of AR47A6.4.2 against another human cancer xenograft model, the antibody was tested on a MCF-7 cancer xenograft model (as disclosed in Ser. No. 11/709,676). AR47A6.4.2 reduced tumor growth in the MCF-7 in vivo prophylactic model of human breast cancer. Treatment with ARIUS antibody AR47A6.4.2 resulted in a marked tumor growth delay. AR47A6.4.2 induced T/C percent values that were lower than 42 percent from day 18 to day 35 of treatment and close to 42 percent up to day 49 (optimal T/C percent value of 10.9 percent at day 18). At day 53, after treatment was terminated, efficacy with treatment of AR47A6.4.2 was still observed with a T/C of 57 percent. At the end of the study (day 91), 2 mice from the AR47A6.4.2 treatment group remained tumor-free. A post-treatment survival benefit was associated with AR47A6.4.2 administration. The buffer control group reached 100 percent mortality by day 85 post-treatment while 33.3 percent of the AR47A6.4.2 mice were still alive at day 91 post-treatment. There were no clinical signs of toxicity throughout the study. In summary, AR47A6.4.2 was well-tolerated, reduced tumor growth and provided a survival benefit in this human breast cancer xenograft model. AR47A6.4.2 has demonstrated efficacy against three different human cancer indications; pancreatic, prostate and breast.

AR47A6.4.2 has demonstrated anti-cancer properties against two different human pancreatic, a prostate and a breast cancer xenograft model. To determine the efficacy of AR47A6.4.2 against another human cancer xenograft model, the antibody was tested on a Colo 205 colon cancer xenograft model (as disclosed in Ser. No. 11/709,676). AR47A6.4.2 inhibited tumor growth in the Colo 205 in vivo prophylactic model of human colorectal adenocarcinoma cells. Treatment with ARIUS antibody AR47A6.4.2 reduced the growth of Colo 205 tumors by 60.2 percent (p=0.0003851, t-test), compared to the buffer-treated group, as determined on day 27, 4 days before the last dose of antibody. There were no obvious clinical signs of toxicity throughout the study. In summary, AR47A6.4.2 was well-tolerated and significantly inhibited the tumor growth in this human colon cancer xenograft model. AR47A6.4.2 has demonstrated efficacy against four different human cancer indications; pancreatic, prostate, breast and colon. Treatment benefits were observed in several well-recognized models of human cancer disease suggesting pharmacologic and pharmaceutical benefits of this antibody for therapy in other mammals, including man. In toto, this data demonstrates that the AR47A6.4.2 antigen is a cancer associated antigen and is expressed on human cancer cells, and is a pathologically relevant cancer target.

As disclosed previously (Ser. No. 11/709,676), biochemical data indicated that the antigen recognized by AR47A6.4.2 is TROP-2. This was supported by studies that showed a monoclonal antibody (clone 77220.11, R&D Systems, Minneapolis, Minn.) reactive against TROP-2 identifies proteins that were bound to AR47A6.4.2 by immunoprecipitation. In addition, AR47A6.4.2 specifically recognized the recombinant form of human TROP-2 by Western Immunoblot. The AR47A6.4.2 epitope does not appear to be carbohydrate dependent but does appear to be conformation dependent. AR47A6.4.2 was also demonstrated to bind to a distinct epitope from another anti-TROP-2 antibody: AR52A301.5.

In order to determine the utility of the AR47A6.4.2 epitope, the expression of AR47A6.4.2 antigen in frozen normal human tissue sections (experiments showed no reactivity of this antibody with formalin fixed tissues) was previously determined (as disclosed in Ser. No. 11/709,676). Binding to 12 human normal organs, ovary, pancreas, thyroid, brain (cerebrum, cerebellum), lung, spleen, uterus, cervix, heart, skin, and skeletal muscle was performed using a human normal tissue screening array (Biochain, CA, USA). The array contained 20 normal human organs; however only 12 of the organs were interpretable after staining. The AR47A6.4.2 antibody showed binding predominantly to epithelial tissues (endothelium of blood vessels, follicular epithelium of thyroid, acinar and ductal epithelium of pancreas, alveolar epithelium of lung, and epidermal keratinocytes of skin). The antibody also showed equivocal binding to lymphoid tissue of the spleen and binding to neural tissue of the brain. Cellular localization was cytoplasmic and membranous with diffuse staining pattern. AR47A6.4.2 showed a similar binding pattern when compared to a research anti-TROP-2 antibody (clone 77220.11).

To further extend the therapeutic benefit of AR47A6.4.2, the frequency and localization of the antigen within various human cancer tissues and their corresponding normal tissue sections (10 colon cancers and 1 normal colon, 7 ovarian cancers and 1 normal ovary, 11 breast cancers and 3 normal breast, 14 lung cancers and 3 normal lung, 13 prostate cancers and 3 normal prostate, and 13 pancreatic cancers and 4 normal pancreas) was also previously determined (as disclosed in Ser. No. 11/709,676). AR47A6.4.2 showed moderate to strong binding to 5/10 (50 percent), 6/7 (86 percent), 10/11 (91 percent), 11/14 (79 percent), 13/13 (100 percent) and 2/13 (15 percent) of colon, ovarian, breast, lung, prostate and pancreatic cancers, respectively. In addition, equivocal to weak binding was observed in 2/10 (20 percent), 1/11 (9 percent), 3/14 (21 percent), and 2/13 (15 percent) colon, breast, lung and pancreatic cancer sections, respectively. In all of the tested tumors, the binding was specific for the tumor cells. For the corresponding normal tissues the antibody showed binding to 0/1, 0/1, 3/3, 3/3, 3/3 and 4/4 of normal colon, ovary, breast, lung, prostate, and pancreatic tissues. However, the binding was predominantly to the epithelial tissues of the normal organs.

IHC studies were previously conducted to characterize the AR47A6.4.2 antigen cross reactivity in frozen normal tissues of various species (as disclosed in Ser. No. 11/709,676). AR47A6.4.2 showed no detectable binding to the tested mouse, rat, guinea pig, goat, sheep, hamster, chicken, cow, horse or pig normal tissues. For the normal rabbit and dog tissues, there was dissimilar binding to that observed in the corresponding human tissues. For the cynomolgus monkey normal tissues, AR47A6.4.2 showed similar tissue specificity as observed in the corresponding human normal tissues for all of the tested organs except for the ovary and testis in which no detectable binding was observed for the cynomolgus monkey sections. For the rhesus monkey normal tissues, AR47A6.4.2 showed similar tissue specificity as observed in the corresponding human normal tissues. It should be noted that the rhesus monkey normal tissue panel was smaller than what was tested for the cynomolgus monkey. Based on the staining profiles, both the cynomolgus and rhesus monkey have similar AR47A6.4.2 antigen distribution to human tissues.

To facilitate production of antibody chimera, the genes encoding the variable regions of both heavy and light chains were separately cloned and sequenced (as previously disclosed in Ser. No. 11/709,676).

The present invention describes the development and use of AR47A6.4.2, chimeric AR47A6.4.2 ((ch)AR47A6.4.2) and humanized variants, (hu)AR47A6.4.2. AR47A6.4.2 was identified by its effect in cytotoxic assays, in tumor growth models and in prolonging survival time in mammals suffering from cancerous disease. This invention represents an advance in the field of cancer treatment in that it describes, for the first time, reagents that bind specifically to an epitope or epitopes present on the target molecule, TROP-2, and that also have in vitro cytotoxic properties, as a naked antibody, against malignant tumor cells but not normal cells, and which also directly mediate, as a naked antibody, inhibition of tumor growth and extension of survival in in vivo models of human cancer. This is an advance in relation to any other previously described anti-TROP-2 antibody, since none have been shown to have similar properties. It also provides an advance in the field since it clearly demonstrates, and for the first time, the direct involvement of TROP-2 in events associated with growth and development of certain types of tumors. It also represents an advance in cancer therapy since it has the potential to display similar anti-cancer properties in human patients. A further advance is that inclusion of these antibodies in a library of anti-cancer antibodies will enhance the possibility of targeting tumors expressing different antigen markers by determination of the appropriate combination of different anti-cancer antibodies, to find the most effective in targeting and inhibiting growth and development of the tumors.

In all, this invention teaches the use of the AR47A6.4.2 antigen as a target for a therapeutic agent, that when administered can reduce the tumor burden of a cancer expressing the antigen in a mammal, and can also lead to a prolonged survival of the treated mammal. This invention also teaches the use of CDMAB (AR47A6.4.2, chimeric AR47A6.4.2 ((ch)AR47A6.4.2) and humanized variants, (hu)AR47A6.4.2), and its derivatives, and antigen binding fragments thereof, and cellular cytotoxicity inducing ligands thereof to target their antigen to reduce the tumor burden of a cancer expressing the antigen in a mammal, and lead to prolonged survival of the treated mammal. Furthermore, this invention also teaches the use of detecting the AR47A6.4.2 antigen in cancerous cells that can be useful for the diagnosis, prediction of therapy, and prognosis of mammals bearing tumors that express this antigen.

Accordingly, it is an objective of the invention to utilize a method for producing cancerous disease modifying antibodies (CDMAB) raised against cancerous cells derived from a particular individual, or one or more particular cancer cell lines, which CDMAB are cytotoxic with respect to cancer cells while simultaneously being relatively non-toxic to non-cancerous cells, in order to isolate hybridoma cell lines and the corresponding isolated monoclonal antibodies and antigen binding fragments thereof for which said hybridoma cell lines are encoded.

It is an additional objective of the invention to teach cancerous disease modifying antibodies, ligands and antigen binding fragments thereof.

It is a further objective of the instant invention to produce cancerous disease modifying antibodies whose cytotoxicity is mediated through antibody dependent cellular toxicity.

It is yet an additional objective of the instant invention to produce cancerous disease modifying antibodies whose cytotoxicity is mediated through complement dependent cellular toxicity.

It is still a further objective of the instant invention to produce cancerous disease modifying antibodies whose cytotoxicity is a function of their ability to catalyze hydrolysis of cellular chemical bonds.

A still further objective of the instant invention is to produce cancerous disease modifying antibodies which are useful for in a binding assay for diagnosis, prognosis, and monitoring of cancer.

Other objects and advantages of this invention will become apparent from the following description wherein are set forth, by way of illustration and example, certain embodiments of this invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 demonstrates the effect of AR47A6.4.2 on tumor growth in a prophylactic human MDA-MB-231 breast cancer model. The vertical dashed lines indicate the period during which the antibody was intraperitoneally administered. Data points represent the mean +/−SEM.

FIG. 2 demonstrates the effect of AR47A6.4.2 on mouse survival in a prophylactic MDA-MB-231 breast cancer model. Data points represent the survival percentage.

FIG. 3 demonstrates the effect of AR47A6.4.2 on mouse body weight in a prophylactic MDA-MB-231 breast adenocarcinoma model. Data points represent the mean +/−SEM.

FIG. 4 demonstrates the effect of AR47A6.4.2 on tumor growth in an established human PL45 pancreatic cancer model in a dose-response manner. The vertical dashed lines indicate the period during which the antibody was intraperitoneally administered. Data points represent the mean +/−SEM.

FIG. 5 demonstrates the effect of AR47A6.4.2 on mouse survival in an established PL45 pancreatic cancer model. Data points represent the survival percentage.

FIG. 6 demonstrates the effect of AR47A6.4.2 on mouse body weight in an established PL45 pancreatic cancer model. Data points represent the mean +/−SEM.

FIG. 7 tabulates an IHC comparison of AR47A6.4.2 on various human tumor and normal tissue sections from different tissue micro arrays.

FIG. 8. Representative micrographs showing the binding pattern on breast tumor tissue obtained with AR47A6.4.2 (A) or the isotype control antibody (B) and on prostate tumor tissue obtained with AR47A6.4.2 (C) or the isotype control antibody (D) and on pancreatic tumor tissue obtained with AR47A6.4.2 (E) or the isotype control antibody (F) from various human tumor tissue microarrays. Magnification is 400× for the breast and pancreatic tumor tissue and 200× for the prostate tumor tissue.

FIG. 9. Representative micrographs showing the binding pattern obtained with AR47A6.4.2 on ovarian tumor tissue (A) or ovarian normal tissue (B). AR47A6.4.2 showed strong binding to the tumor but not the corresponding normal tissue. Magnification is 200×.

FIG. 10. List of kinases whose phosphorylation is affected by treatment of BxPC-3 cells treated with AR47A6.4.2 followed by serum and supplement stimulation.

FIG. 11. List of secreted angiogenic factors affected by the treatment of BxPC-3 cells treated with AR47A6.4.2.

FIG. 12 demonstrates in vitro CDC activity of AR47A6.4.2 on two different human pancreatic cancer cell lines; PL45 and BxPC-3.

FIG. 13. Binding of AR47A6.4.2 to CLIPS peptides that were synthesized based on the TROP-2 amino acid sequence.

FIG. 14. Amino acid sequence of TROP-2. The discontinuous epitope recognized by AR47A6.4.2 is contained within the underlined sequences. Amino acid positions 1-274 represent the extracellular portion of TROP-2; amino acid positions 275-290 represent the transmembrane portion of TROP-2 and amino acid positions 291-232 represent the intracellular portion of TROP-2.

FIG. 15. Primers used in the PCR amplification of light chain.

FIG. 16. Primers used in the PCR amplification of heavy chain.

FIG. 17. Mouse AR47A6.4.2 VH Sequence.

FIG. 18. Mouse AR47A6.4.2 VL Sequence.

FIG. 19. Oligonucleotides used for the generation of chimeric and variant humanized AR47A6.4.2 VH sequences.

FIG. 20. Oligonucleotides used for the generation of chimeric and variant humanized AR47A6.4.2 VL sequences.

FIG. 21. Light chain and heavy chain expression vectors.

FIGS. 22A, 22B and 22C. Humanized AR47A6.4.2 VH variants. CDRs are underlined.

FIGS. 23A, 23B and 23C. Humanized AR47A6.4.2 VL variants. CDRs are underlined.

FIG. 24. Activities of humanized AR47A6.4.2 VH and VL variants.

FIG. 25. Summary of the binding affinity association rate constants (Ka) and dissociation rate constants (Kd) of murine AR47A6.4.2 and various variants of (hu)AR47A.6.4.2 to rhTROP-2.

### DETAILED DESCRIPTION OF THE INVENTION

In general, the following words or phrases have the indicated definition when used in the summary, description, examples, and claims.

The term “antibody” is used in the broadest sense and specifically covers, for example, single monoclonal antibodies (including agonist, antagonist, and neutralizing antibodies, de-immunized, murine, chimeric or humanized antibodies), antibody compositions with polyepitopic specificity, single-chain antibodies, diabodies, triabodies, immunoconjugates and antibody fragments (see below).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma (murine or human) method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Antibody fragments” comprise a portion of an intact antibody, preferably comprising the antigen-binding or variable region thereof. Examples of antibody fragments include less than full length antibodies, Fab, Fab′, F(ab′)₂, and Fv fragments; diabodies; linear antibodies; single-chain antibody molecules; single-chain antibodies, single domain antibody molecules, fusion proteins, recombinant proteins and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol; 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., Eur. J. Immunol. 24:2429 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved-directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 2632 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH I) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “triabodies” or “trivalent trimers” refers to the combination of three single chain antibodies. Triabodies are constructed with the amino acid terminus of a V_{L} or V_{H} domain, i.e., without any linker sequence. A triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. Isolated antibody includes the antibody in situ within recombinant cells since at least one component of the antibody's natural environment will not be present. Ordinarily, however, isolated antibody will be prepared by at least one purification step.

An antibody “which binds” an antigen of interest, e.g. TROP-2 antigen, is one capable of binding that antigen with sufficient affinity such that the antibody is useful as a therapeutic or diagnostic agent in targeting a cell expressing the antigen. Where the antibody is one which binds TROP-2, it will usually preferentially bind TROP-2 as opposed to other receptors, and does not include incidental binding such as non-specific Fc contact, or binding to post-translational modifications common to other antigens and may be one which does not significantly cross-react with other proteins. Methods, for the detection of an antibody that binds an antigen of interest, are well known in the art and can include but are not limited to assays such as FACS, cell ELISA and Western blot.

As used herein, the expressions “cell”, “cell line”, and “cell culture” are used interchangeably, and all such designations include progeny. It is also understood that all progeny may not be precisely identical in DNA content, due to deliberate or inadvertent mutations. Mutant progeny that have the same function or biological activity as screened for in the originally transformed cell are included. It will be clear from the context where distinct designations are intended.

“Treatment or treating” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Hence, the mammal to be treated herein may have been diagnosed as having the disorder or may be predisposed or susceptible to the disorder.

The terms “cancer” and “cancerous” refer to or describe the physiological condition in mammals that is typically characterized by unregulated cell growth or death. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Mammal” for purposes of treatment refers to any animal classified as a mammal, including humans, mice, SCID or nude mice or strains of mice, domestic and farm animals, and zoo, sports, or pet animals, such as sheep, dogs, horses, cats, cows, etc. Preferably, the mammal herein is human.

“Oligonucleotides” are short-length, single- or double-stranded polydeoxynucleotides that are chemically synthesized by known methods (such as phosphotriester, phosphite, or phosphoramidite chemistry, using solid phase techniques such as described in EP 266,032, published 4 May 1988, or via deoxynucleoside H-phosphonate intermediates as described by Froehler et al., Nucl. Acids Res., 14:5399-5407, 1986. They are then purified on polyacrylamide gels.

In accordance with the present invention, “humanized” and/or “chimeric” forms of non-human (e.g. murine) immunoglobulins refer to antibodies which contain specific chimeric immunoglobulins, immunoglobulin chains or fragments thereof (such as Fv, Fab, Fab′, F(ab′)₂ or other antigen-binding subsequences of antibodies) which results in the decrease of a human anti-mouse antibody (HAMA), human anti-chimeric antibody (HACA) or a human anti-human antibody (HAHA) response, compared to the original antibody, and contain the requisite portions (e.g. CDR(s), antigen binding region(s), variable domain(s) and so on) derived from said non-human immunoglobulin, necessary to reproduce the desired effect, while simultaneously retaining binding characteristics which are comparable to said non-human immunoglobulin. For the most part, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“De-immunized” antibodies are immunoglobulins that are non-immunogenic, or less immunogenic, to a given species. De-immunization can be achieved through structural alterations to the antibody. Any de-immunization technique known to those skilled in the art can be employed. One suitable technique for de-immunizing antibodies is described, for example, in WO 00/34317 published Jun. 15, 2000.

An antibody which induces “apoptosis” is one which induces programmed cell death by any means, illustrated by but not limited to binding of annexin V, caspase activity, fragmentation of DNA, cell shrinkage, dilation of endoplasmic reticulum, cell fragmentation, and/or formation of membrane vesicles (called apoptotic bodies).

As used herein “antibody induced cytotoxicity” is understood to mean the cytotoxic effect derived from the hybridoma supernatant or antibody produced by the hybridoma deposited with the IDAC as accession number 141205-05, a humanized antibody of the isolated monoclonal antibody produced by the hybridoma deposited with the IDAC as accession number 141205-05, a chimeric antibody of the isolated monoclonal antibody produced by the hybridoma deposited with the IDAC as accession number 141205-05, antigen binding fragments, or antibody ligands thereof, which effect is not necessarily related to the degree of binding.

Throughout the instant specification, hybridoma cell lines, as well as the isolated monoclonal antibodies which are produced therefrom, are alternatively referred to by their internal designation, AR47A6.4.2 (murine), (ch)AR47A6.4.2 (chimeric), (hu)AR47A6.4.2 (humanized) or Depository Designation, IDAC 141205-05.

As used herein “antibody-ligand” includes a moiety which exhibits binding specificity for at least one epitope of the target antigen, and which may be an intact antibody molecule, antibody fragments, and any molecule having at least an antigen-binding region or portion thereof (i.e., the variable portion of an antibody molecule), e.g., an Fv molecule, Fab molecule, Fab′ molecule, F(ab′).sub.2 molecule, a bispecific antibody, a fusion protein, or any genetically engineered molecule which specifically recognizes and binds at least one epitope of the antigen bound by the isolated monoclonal antibody produced by the hybridoma cell line designated as IDAC 141205-05 (the IDAC 141205-05 antigen), a humanized antibody of the isolated monoclonal antibody produced by the hybridoma deposited with the IDAC as accession number 141205-05, a chimeric antibody of the isolated monoclonal antibody produced by the hybridoma deposited with the IDAC as accession number 141205-05 and antigen binding fragments.

As used herein “cancerous disease modifying antibodies” (CDMAB) refers to monoclonal antibodies which modify the cancerous disease process in a manner which is beneficial to the patient, for example by reducing tumor burden or prolonging survival of tumor bearing individuals, and antibody-ligands thereof.

A “CDMAB related binding agent”, in its broadest sense, is understood to include, but is not limited to, any form of human or non-human antibodies, antibody fragments, antibody ligands, or the like, which competitively bind to at least one CDMAB target epitope.

A “competitive binder” is understood to include any form of human or non-human antibodies, antibody fragments, antibody ligands, or the like which has binding affinity for at least one CDMAB target epitope.

Tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents but recur up to five years, sometimes up to ten years or longer after treatment is discontinued.

Tumors that can be treated include tumors that are not vascularized, or not yet substantially vascularized, as well as vascularized tumors. Examples of solid tumors, which can be accordingly treated, include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma.

As used herein “antigen-binding region” means a portion of the molecule which recognizes the target antigen.

As used herein “competitively inhibits” means being able to recognize and bind a determinant site to which the monoclonal antibody produced by the hybridoma cell line designated as IDAC 141205-05, (the IDAC 141205-05 antibody), a humanized antibody of the isolated monoclonal antibody produced by the hybridoma deposited with the IDAC as accession number 141205-05, a chimeric antibody of the isolated monoclonal antibody produced by the hybridoma deposited with the IDAC as accession number 141205-05, antigen binding fragments, or antibody ligands thereof, is directed using conventional reciprocal antibody competition assays. (Belanger L., Sylvestre C. and Dufour D. (1973), Enzyme linked immunoassay for alpha fetoprotein by competitive and sandwich procedures. Clinica Chimica Acta 48, 15).

As used herein “target antigen” is the IDAC 141205-05 antigen or portions thereof.

As used herein, an “immunoconjugate” means any molecule or CDMAB such as an antibody chemically or biologically linked to cytotoxins, radioactive agents, cytokines, interferons, target or reporter moieties, enzymes, toxins, anti-tumor drugs or therapeutic agents. The antibody or CDMAB may be linked to the cytotoxin, radioactive agent, cytokine, interferon, target or reporter moiety, enzyme, toxin, anti-tumor drug or therapeutic agent at any location along the molecule so long as it is able to bind its target. Examples of immunoconjugates include antibody toxin chemical conjugates and antibody-toxin fusion proteins.

Radioactive agents suitable for use as anti-tumor agents are known to those skilled in the art. For example, 131I or 211At is used. These isotopes are attached to the antibody using conventional techniques (e.g. Pedley et al., Br. J. Cancer 68, 69-73 (1993)). Alternatively, the anti-tumor agent which is attached to the antibody is an enzyme which activates a prodrug. A prodrug may be administered which will remain in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques. Interferons may also be used.

As used herein, a “fusion protein” means any chimeric protein wherein an antigen binding region is connected to a biologically active molecule, e.g., toxin, enzyme, fluorescent proteins, luminescent marker, polypeptide tag, cytokine, interferon, target or reporter moiety or protein drug.

The invention further contemplates CDMAB of the present invention to which target or reporter moieties are linked. Target moieties are first members of binding pairs. Anti-tumor agents, for example, are conjugated to second members of such pairs and are thereby directed to the site where the antigen-binding protein is bound. A common example of such a binding pair is avidin and biotin. In a preferred embodiment, biotin is conjugated to the target antigen of the CDMAB of the present invention, and thereby provides a target for an anti-tumor agent or other moiety which is conjugated to avidin or streptavidin. Alternatively, biotin or another such moiety is linked to the target antigen of the CDMAB of the present invention and used as a reporter, for example in a diagnostic system where a detectable signal-producing agent is conjugated to avidin or streptavidin.

Detectable signal-producing agents are useful in vivo and in vitro for diagnostic purposes. The signal producing agent produces a measurable signal which is detectable by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible region, and can be substrates or degradation products of enzyme catalyzed reactions.

Moreover, included within the scope of the present invention is use of the present CDMAB in vivo and in vitro for investigative or diagnostic methods, which are well known in the art. In order to carry out the diagnostic methods as contemplated herein, the instant invention may further include kits, which contain CDMAB of the present invention. Such kits will be useful for identification of individuals at risk for certain type of cancers by detecting over-expression of the CDMAB's target antigen on cells of such individuals.

### Diagnostic Assay Kits

It is contemplated to utilize the CDMAB of the present invention in the form of a diagnostic assay kit for determining the presence of a tumor. The tumor will generally be detected in a patient based on the presence of one or more tumor-specific antigens, e.g. proteins and/or polynucleotides which encode such proteins in a biological sample, such as blood, sera, urine and/or tumor biopsies, which samples will have been obtained from the patient.

The proteins function as markers which indicate the presence or absence of a particular tumor, for example a colon, breast, lung or prostate tumor. It is further contemplated that the antigen will have utility for the detection of other cancerous tumors. Inclusion in the diagnostic assay kits of binding agents comprised of CDMABs of the present invention, or CDMAB related binding agents, enables detection of the level of antigen that binds to the agent in the biological sample. Polynucleotide primers and probes may be used to detect the level of mRNA encoding a tumor protein, which is also indicative of the presence or absence of a cancer. In order for the binding assay to be diagnostic, data will have been generated which correlates statistically significant levels of antigen, in relation to that present in normal tissue, so as to render the recognition of binding definitively diagnostic for the presence of a cancerous tumor. It is contemplated that a plurality of formats will be useful for the diagnostic assay of the present invention, as are known to those of ordinary skill in the art, for using a binding agent to detect polypeptide markers in a sample. For example, as illustrated in Harlow and Lane, Antibodies: A Laboratory Manual, Cold Spring Harbor Laboratory, 1988. Further contemplated are any and all combinations, permutations or modifications of the afore-described diagnostic assay formats.

The presence or absence of a cancer in a patient will typically be determined by (a) contacting a biological sample obtained from a patient with a binding agent; (b) detecting in the sample a level of polypeptide that binds to the binding agent; and (c) comparing the level of polypeptide with a predetermined cut-off value.

In an illustrative embodiment, it is contemplated that the assay will involve the use of a CDMAB based binding agent immobilized on a solid support to bind to and remove the polypeptide from the remainder of the sample. The bound polypeptide may then be detected using a detection reagent that contains a reporter group and specifically binds to the binding agent/polypeptide complex. Illustrative detection reagents may include a CDMAB based binding agent that specifically binds to the polypeptide or an antibody or other agent that specifically binds to the binding agent, such as an anti-immunoglobulin, protein G, protein A or a lectin. In an alternative embodiment, it is contemplated that a competitive assay may be utilized, in which a polypeptide is labeled with a reporter group and allowed to bind to the immobilized binding agent after incubation of the binding agent with the sample. Indicative of the reactivity of the sample with the immobilized binding agent, is the extent to which components of the sample inhibit the binding of the labeled polypeptide to the binding agent. Suitable polypeptides for use within such assays include full length tumor-specific proteins and/or portions thereof, to which the binding agent has binding affinity.

The diagnostic kit will be provided with a solid support which may be in the form of any material known to those of ordinary skill in the art to which the protein may be attached. Suitable examples may include a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

It is contemplated that the binding agent will be immobilized on the solid support using a variety of techniques known to those of skill in the art, which are amply described in the patent and scientific literature. The term “immobilization” refers to both noncovalent association, such as adsorption, and covalent attachment, which, in the context of the present invention, may be a direct linkage between the agent and functional groups on the support, or may be a linkage by way of a cross-linking agent. In a preferred, albeit non-limiting embodiment, immobilization by adsorption to a well in a microtiter plate or to a membrane is preferable. Adsorption may be achieved by contacting the binding agent, in a suitable buffer, with the solid support for a suitable amount of time. The contact time may vary with temperature, and will generally be within a range of between about 1 hour and about 1 day.

Covalent attachment of binding agent to a solid support would ordinarily be accomplished by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12 A13).

It is further contemplated that the diagnostic assay kit will take the form of a two-antibody sandwich assay. This assay may be performed by first contacting an antibody, e.g. the instantly disclosed CDMAB that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

In a specific embodiment, it is contemplated that once the antibody is immobilized on the support as described above, the remaining protein binding sites on the support will be blocked, via the use of any suitable blocking agent known to those of ordinary skill in the art, such as bovine serum albumin or Tween 20™ (Sigma Chemical Co., St. Louis, Mo.). The immobilized antibody would then be incubated with the sample, and polypeptide would be allowed to bind to the antibody. The sample could be diluted with a suitable diluent, such as phosphate-buffered saline (PBS) prior to incubation. In general, an appropriate contact time (i.e., incubation time) would be selected to correspond to a period of time sufficient to detect the presence of polypeptide within a sample obtained from an individual with the specifically selected tumor. Preferably, the contact time is sufficient to achieve a level of binding that is at least about 95 percent of that achieved at equilibrium between bound and unbound polypeptide. Those of ordinary skill in the art will recognize that the time necessary to achieve equilibrium may be readily determined by assaying the level of binding that occurs over a period of time.

It is further contemplated that unbound sample would then be removed by washing the solid support with an appropriate buffer. The second antibody, which contains a reporter group, would then be added to the solid support. Incubation of the detection reagent with the immobilized antibody-polypeptide complex would then be carried out for an amount of time sufficient to detect the bound polypeptide. Subsequently, unbound detection reagent would then be removed and bound detection reagent would be detected using the reporter group. The method employed for detecting the reporter group is necessarily specific to the type of reporter group selected, for example for radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

In order to utilize the diagnostic assay kit of the present invention to determine the presence or absence of a cancer, such as prostate cancer, the signal detected from the reporter group that remains bound to the solid support would generally be compared to a signal that corresponds to a predetermined cut-off value. For example, an illustrative cut-off value for the detection of a cancer may be the average mean signal obtained when the immobilized antibody is incubated with samples from patients without the cancer. In general, a sample generating a signal that is about three standard deviations above the predetermined cut-off value would be considered positive for the cancer. In an alternate embodiment, the cut-off value might be determined by using a Receiver Operator Curve, according to the method of Sackett et al., Clinical Epidemiology. A Basic Science for Clinical Medicine, Little Brown and Co., 1985, p. 106-7. In such an embodiment, the cut-off value could be determined from a plot of pairs of true positive rates (i.e., sensitivity) and false positive rates (100 percent-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

It is contemplated that the diagnostic assay enabled by the kit will be performed in either a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound will be immersed in a solution containing the sample. The sample migrates along the membrane through a region containing second binding agent and to the area of immobilized binding agent. Concentration of the second binding agent at the area of immobilized antibody indicates the presence of a cancer. Generation of a pattern, such as a line, at the binding site, which can be read visually, will be indicative of a positive test. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in the instant diagnostic assay are the instantly disclosed antibodies, antigen-binding fragments thereof, and any CDMAB related binding agents as herein described. The amount of antibody immobilized on the membrane will be any amount effective to produce a diagnostic assay, and may range from about 25 nanograms to about 1 microgram. Typically such tests may be performed with a very small amount of biological sample.

Additionally, the CDMAB of the present invention may be used in the laboratory for research due to its ability to identify its target antigen.

In order that the invention herein described may be more fully understood, the following description is set forth.

The present invention provides CDMAB (i.e., IDAC 141205-05 CDMAB, a humanized antibody of the isolated monoclonal antibody produced by the hybridoma deposited with the IDAC as accession number 141205-05, a chimeric antibody of the isolated monoclonal antibody produced by the hybridoma deposited with the IDAC as accession number 141205-05, antigen binding fragments, or antibody ligands thereof) which specifically recognize and bind the IDAC 141205-05 antigen.

The CDMAB of the isolated monoclonal antibody produced by the hybridoma deposited with the IDAC as accession number 141205-05 may be in any form as long as it has an antigen-binding region which competitively inhibits the immunospecific binding of the isolated monoclonal antibody produced by hybridoma IDAC 141205-05 to its target antigen. Thus, any recombinant proteins (e.g., fusion proteins wherein the antibody is combined with a second protein such as a lymphokine or a tumor inhibitory growth factor) having the same binding specificity as the IDAC 141205-05 antibody fall within the scope of this invention.

In one embodiment of the invention, the CDMAB is the IDAC 141205-05 antibody.

In other embodiments, the CDMAB is an antigen binding fragment which may be a Fv molecule (such as a single-chain Fv molecule), a Fab molecule, a Fab′ molecule, a F(ab′)2 molecule, a fusion protein, a bispecific antibody, a heteroantibody or any recombinant molecule having the antigen-binding region of the IDAC 141205-05 antibody. The CDMAB of the invention is directed to the epitope to which the IDAC 141205-05 monoclonal antibody is directed.

The CDMAB of the invention may be modified, i.e., by amino acid modifications within the molecule, so as to produce derivative molecules. Chemical modification may also be possible. Modification by direct mutation, methods of affinity maturation, phage display or chain shuffling may also be possible.

Affinity and specificity can be modified or improved by mutating CDR and/or phenylalanine tryptophan (FW) residues and screening for antigen binding sites having the desired characteristics (e.g., Yang et al., J. Mol. Biol., (1995) 254: 392-403). One way is to randomize individual residues or combinations of residues so that in a population of otherwise identical antigen binding sites, subsets of from two to twenty amino acids are found at particular positions. Alternatively, mutations can be induced over a range of residues by error prone PCR methods (e.g., Hawkins et al., J. Mol. Biol., (1992) 226: 889-96). In another example, phage display vectors containing heavy and light chain variable region genes can be propagated in mutator strains of E. coli (e.g., Low et al., J. Mol. Biol., (1996) 250: 359-68). These methods of mutagenesis are illustrative of the many methods known to one of skill in the art.

Another manner for increasing affinity of the antibodies of the present invention is to carry out chain shuffling, where the heavy or light chain are randomly paired with other heavy or light chains to prepare an antibody with higher affinity. The various CDRs of the antibodies may also be shuffled with the corresponding CDRs in other antibodies.

Derivative molecules would retain the functional property of the polypeptide, namely, the molecule having such substitutions will still permit the binding of the polypeptide to the IDAC 141205-05 antigen or portions thereof.

These amino acid substitutions include, but are not necessarily limited to, amino acid substitutions known in the art as “conservative”.

For example, it is a well-established principle of protein chemistry that certain amino acid substitutions, entitled “conservative amino acid substitutions,” can frequently be made in a protein without altering either the conformation or the function of the protein.

Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

### EXAMPLE 1

### In Vivo Tumor Experiment with Human MDA-MB-231 Breast Cancer Cells

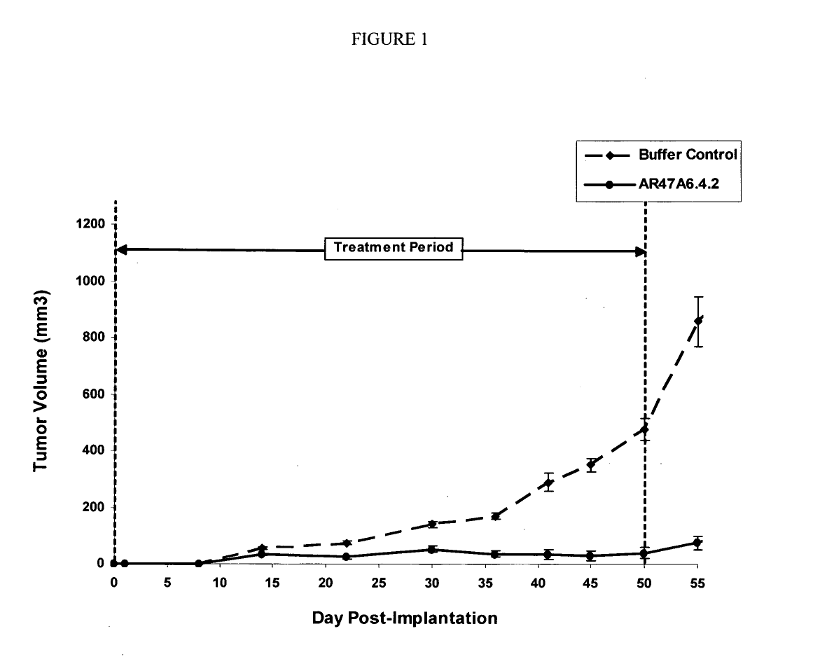

AR47A6.4.2 had previously demonstrated (as disclosed in Ser. No. 11/709,676) efficacy in a MCF-7 human breast cancer xenograft model. To extend this finding AR47A6.4.2 was tested in a MDA-MB-231 human breast cancer xenograft model which differs from the MCF-7 model and is Her2/neu negative, estrogen and progesterone receptor negative. With reference to FIGS. 1, 2 and 3, 8 to 10 week old female SCID mice were implanted with 5 million human breast cancer cells (MDA-MB-231) in 100 microliters PBS solution injected subcutaneously in the right flank of each mouse. The mice were randomly divided into 2 treatment groups of 10. One day after implantation, 20 mg/kg of AR47A6.4.2 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered once per week for the first two weeks and twice per week for another 3 weeks. Tumor growth was measured once per week with calipers. The treatment was completed after 8 doses of antibody. Body weights of the animals were recorded at the same time as tumor measurement. All animals were euthanized according to CCAC guidelines at the end of the study once they had reached endpoint.

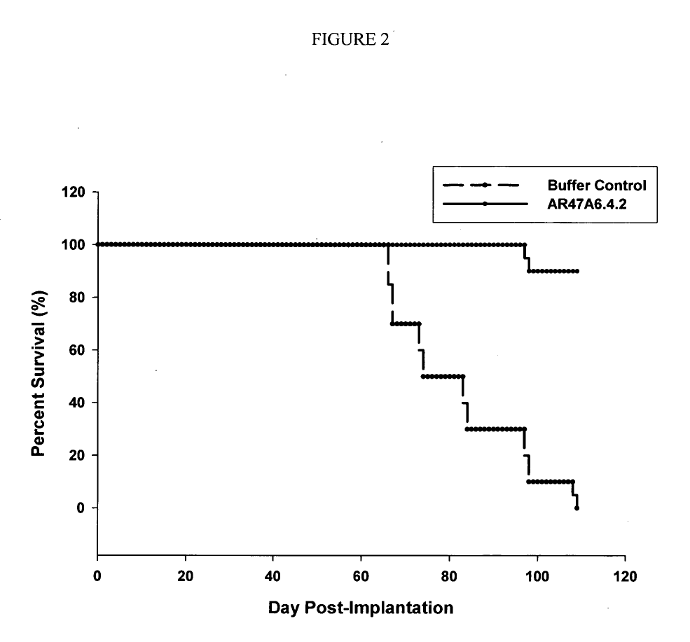

AR47A6.4.2 significantly inhibited tumor growth in the MDA-MB-231 in vivo prophylactic model of human breast cancer. Treatment with ARIUS antibody AR47A6.4.2 reduced the growth of MDA-MB-231 tumors by 91.9 percent (p<0.00001, t-test), compared to the buffer treated group, as determined on day 55, 5 days after the last dose of antibody (FIG. 1). All mice in the control group were removed from the study, due to reaching endpoint, at day 108, 58 days after the last dose of antibody. However, 90 percent of the mice in AR47A6.4.2-treated group were still alive at that time (FIG. 2).

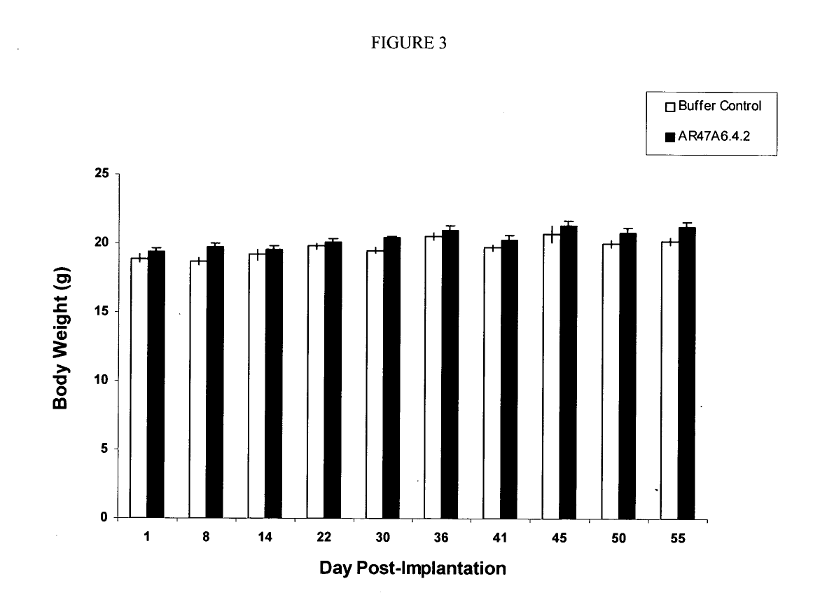

There were no obvious clinical signs of toxicity throughout the study. Body weight measured at weekly intervals was a surrogate for well-being and failure to thrive. The mean body weight increased in all groups over the duration of the study (FIG. 3). The mean weight gain between day 0 and day 55 was 1.3 g (6.9 percent) in the control group and 1.8 g (9.3 percent) in the AR47A6.4.2-treated group. There were no significant differences between the groups during the treatment period. In summary, AR47A6.4.2 was well-tolerated and significantly inhibited the tumor growth in a human breast cancer xenograft model.

### EXAMPLE 2

### In Vivo Tumor Experiment with Human PL45 Pancreatic Cancer Cells

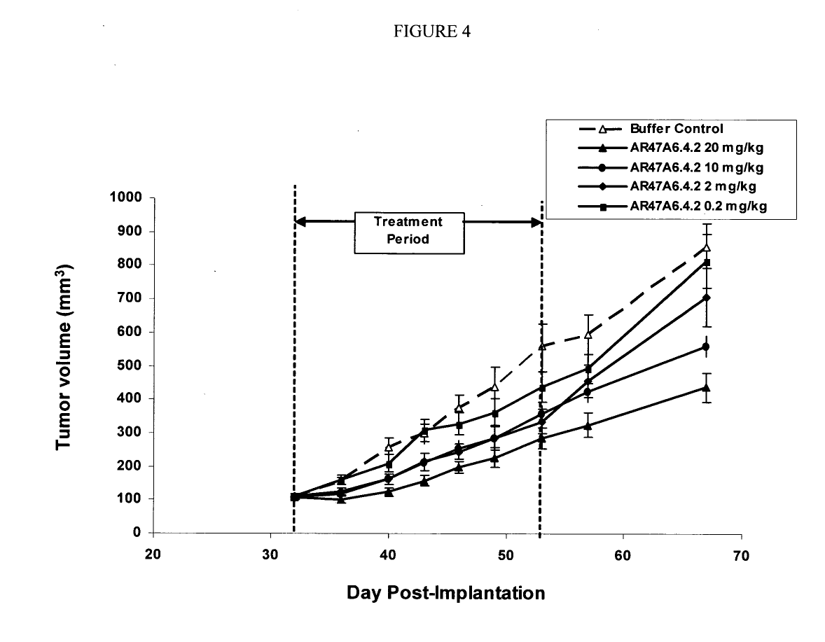

AR47A6.4.2 had previously demonstrated (as disclosed in Ser. No. 11/709,676) efficacy in a preventative PL45 human pancreatic cancer xenograft model. To determine effective dose levels AR47A6.4.2 was tested in an established PL45 model at various doses. With reference to FIGS. 4, 5, and 6, 8 to 10 week old female SCID mice were implanted with 4 million human pancreatic cancer cells (PL45) in 100 microliters PBS solution injected subcutaneously in the scruff of the neck. The mice were randomly divided into 5 treatment groups of 10 when the average mouse tumor volume reached approximately 100 mm³. On day 32 after implantation, 20, 10, 2, or 0.2 mg/kg of AR47A6.4.2 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for the duration of the study. Tumor growth was measured about every 4-7 days with calipers. The study was completed after 10 doses of antibody. Body weights of the animals were recorded once per week for the duration of the study. All animals were euthanized according to CCAC guidelines at the end of the study once they had reached endpoint.

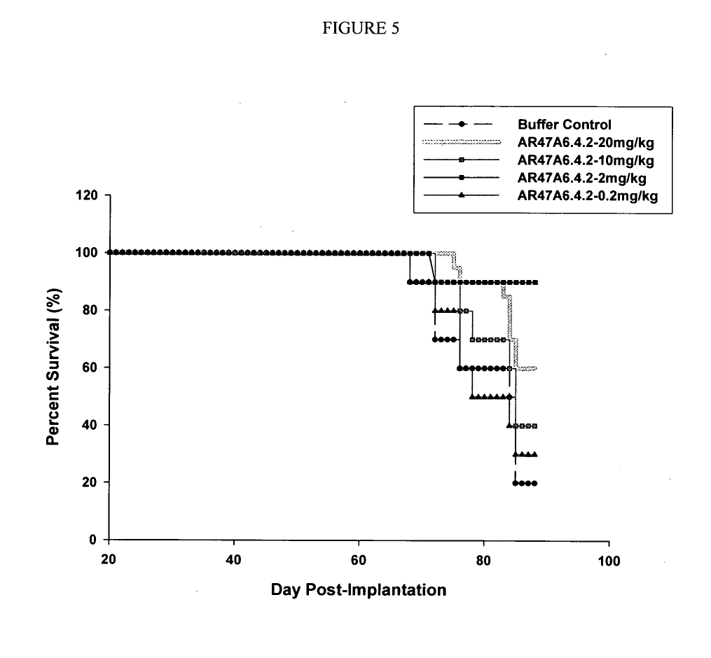

AR47A6.4.2 demonstrated dose-dependent tumor growth inhibition in the PL45 in vivo established model of human pancreatic cancer. Treatment with ARIUS antibody AR47A6.4.2 reduced the growth of PL45 tumors by 48.9 percent (p=0.0001, t-test), 34.6 percent (p=0.0011, t-test), 17.4 percent (p=0.1938, t-test) and 4.7 percent (p=0.7065, t-test) at a dose of 20, 10, 2 and 0.2 mg/kg respectively, compared to the buffer treated group, as determined at day 67, 14 days after last dose of treatment (FIG. 4). This was when almost all mice in the control and antibody-treated groups were still alive. The survival for all groups was monitored until day 88, 35 days after the last dose of treatment. At this time point, only 20 percent ( 2/10) of the mice in the control group were still alive while 60 percent ( 6/10), 40 percent ( 4/10) and 90 percent ( 9/10) of the mice in the AR47A6.4.2-treated group at doses of 20, 10, and 2 mg/kg respectively were still alive (FIG. 5).

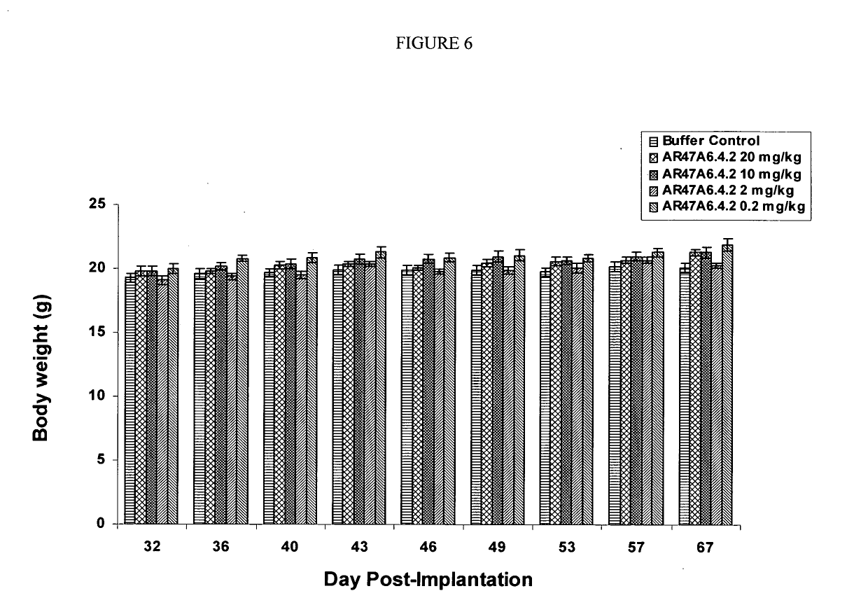

There were no obvious clinical signs of toxicity throughout the study. Body weight measured at weekly intervals was a surrogate for well-being and failure to thrive. The mean body weight increased in all groups over the duration of the study (FIG. 6). The mean weight gain between day 32 and day 67 was 0.8 g (4.1 percent) in the control group and 1.5 g (7.6 percent), 1.5 g (7.6 percent), 1.2 g (6.3 percent) or 1.9 g (9.5 percent) in the AR47A6.4.2-treated group at doses of 20, 10, 2 and 0.2 mg/kg, respectively. There was no significant difference between the groups during the treatment period.

In summary, AR47A6.4.2 was well-tolerated and significantly inhibited the tumor growth in a dose dependent manner in this established human pancreatic cancer xenograft model at 20 and 10 mg/kg. Mice in the AR47A6.4.2-treated group at doses greater than 2 mg/kg also demonstrated a significant survival benefit. In toto, this data demonstrates that AR47A6.4.2 is effective in the treatment of human cancer in a dose dependent manner.

### EXAMPLE 3

### Human Normal and Multi-Tumor Tissue Staining

Additional IHC studies (previous studies were disclosed in Ser. No. 11/709,676) were conducted to further characterize the AR47A6.4.2 antigen prevalence in human cancers. Slides were transferred from −80° C. to −20° C. After one hour, the slides were postfixed for 10 minutes in cold (−20° C.) acetone and then allowed to come to room temperature. Slides were rinsed in 4° C. cold phosphate buffered saline (PBS) 3 times for 2 minutes each followed by blocking endogenous peroxidase activity with washing in 3 percent hydrogen peroxide for 10 minutes. Slides were then rinsed in PBS 3 times for 5 minutes followed by incubation in Universal blocking solution (Dako, Toronto, Ontario) for 5 minutes at room temperature. AR47A6.4.2, anti-human muscle actin (Clone HHF35, Dako, Toronto, Ontario), anti-cytokeratin 7 clone OV-TL 12/30 (Dako, Toronto, Ontario), anti-TROP-2 clone 77220.11 (R&D System Inc., MN, USA) or isotype control antibody (directed towards Aspergillus niger glucose oxidase, an enzyme which is neither present nor inducible in mammalian tissues; Dako, Toronto, Ontario) were diluted in antibody dilution buffer (Dako, Toronto, Ontario) to its working concentration of 5 micrograms/mL for each antibody, except for anti-actin which was 0.5 microgram/mL, anti-cytokeratin 7 was ready to use and commercial anti-TROP-2 was 1 microgram/mL, and incubated for 1 hour at room temperature. The slides were washed with PBS 3 times for 2 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrachloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, ON), the slides were dehydrated with graded ethanols (75-100 percent) and cleared with xylene. Using mounting media (Dako Faramount, Toronto, Ontario) the slides were coverslipped. For the pancreatic array (Tri Star, Rockville, Md.) the same protocol was followed except for the following modifications. The tissue sections were initially air dried at room temperature for 2 hours and air dried again for 30 minutes after fixation with acetone. The endogenous hydrogen peroxide was blocked using 3 percent hydrogen peroxide in methanol for 15 minutes; this step was done after the primary antibody incubation.

Slides were microscopically examined using an Axiovert 200 (Ziess Canada, Toronto, ON) and digital images acquired and stored using Northern Eclipse Imaging Software (Mississauga, ON). Results were read, scored and interpreted by a histopathologist.

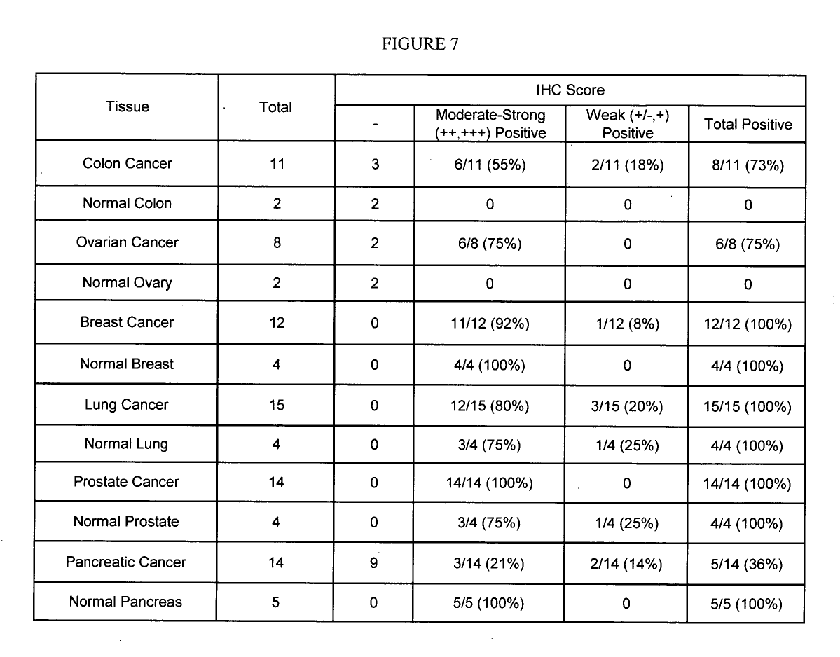

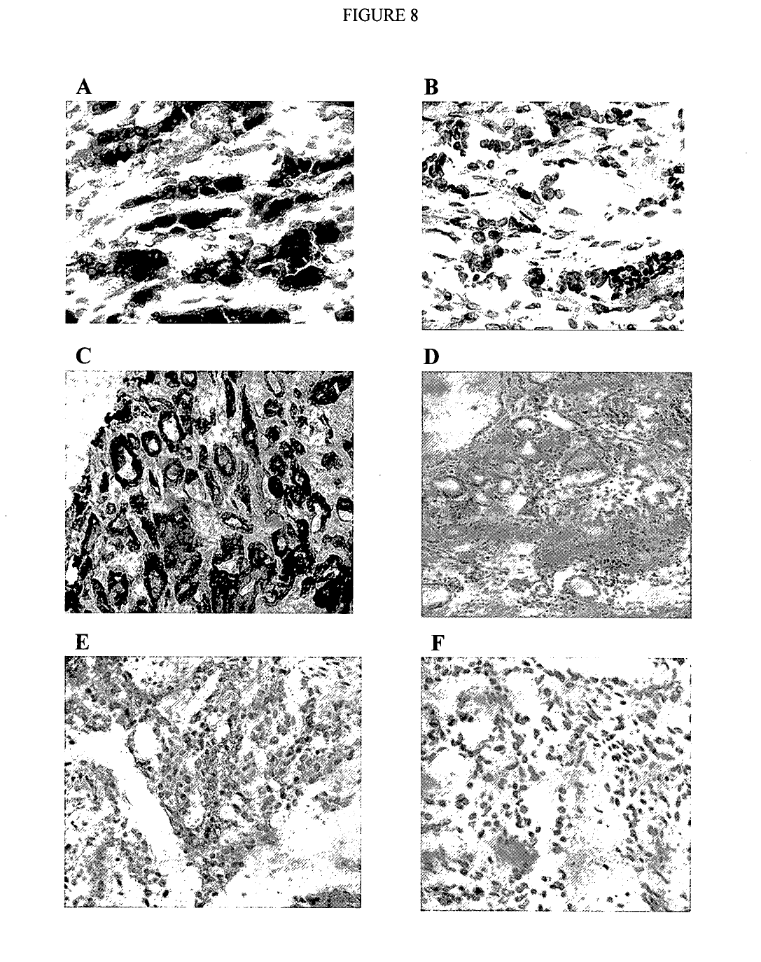

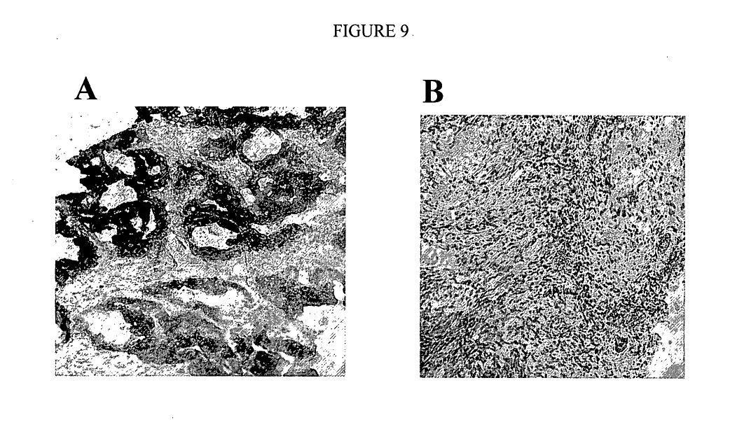

FIG. 7 presents a summary of the results of AR47A6.4.2 staining of panels of human tumors and corresponding normal tissues (11 colon cancers and 2 normal colon, 8 ovarian cancers and 2 normal ovary, 12 breast cancers and 4 normal breast, 15 lung cancers and 4 normal lung, 14 prostate cancers and 4 normal prostate and 14 pancreatic cancers and 5 normal pancreas). These tissues were distributed on four tissue microarrays (Tri Star, Rockville, Md.). The antibody showed moderate to strong binding to 6/11 (55 percent), 6/8 (75 percent), 11/12 (92 percent), 12/15 (80 percent), 14/14 (100 percent) and 3/14 (21 percent) of colon, ovarian, breast, lung, prostate and pancreatic cancers, respectively (FIGS. 8 and 9). In addition, equivocal to weak binding was observed in 2/11 (18 percent), 1/12 (8 percent), 3/15 (20 percent) and 2/14 (14 percent) to colon, breast, lung and pancreatic cancers, respectively. The binding was specific to tumor cells in all tested tumors with over expression in tumors versus normal in some tissues. For corresponding normal tissues the antibody showed binding to 0/2, 0/2, 4/4, 4/4, 4/4 and 5/5 of normal colon, ovary, breast, lung, prostate and pancreatic tissues (FIG. 9). The binding was predominantly to epithelial tissues of those organs. Anti-cytokeratin-7 or anti-actin, used as a positive antibody control, showed the expected positive binding to epithelial tissues and muscular tissues, respectively. The IgG isotype negative control showed negative binding to the tested tissues.

### EXAMPLE 4

### Phospho-MAPK (Mitogen-Activated Protein Kinase) Proteome Profiler Blots

To identify intracellular signaling molecules affected by AR47A6.4.2 treatment, lysates from cells treated with AR47A6.4.2 were screened using a proteome profiler human phospho-MAPK antibody array (ARY002, R&D Systems Inc., Minneapolis, Minn.).

### Treatment and Preparation of Cells

Previous work (as disclosed in Ser. No. 11/709,676) demonstrated in vivo efficacy of AR47A6.4.2 in a pancreatic cancer xenograft model using BxPC-3 cells grown in severe combined immunodeficient (SCID) mice. Accordingly, screening for activation of intracellular signaling molecules was done using the BxPC-3 cell line. BxPC-3 cells were grown to near confluence, washed with phosphate buffered saline (PBS) and then starved in serum and supplement-deficient media for 4 hours at 37° C. After this, AR47A6.4.2 (20 micrograms/mL) or 8A3B.6 (isotype control; IgG2a) (20 micrograms/mL) was added to the cells and allowed to bind for 20 minutes at 4° C. Cells were then stimulated by adding fetal bovine serum (FBS), L-glutamine and sodium pyruvate to the cells to give a final concentration of 10 percent FBS, 1 percent L-glutamine, and 1 percent sodium pyruvate. The cells were placed in an incubator at 37° C. and the cell lysate was collected 1 hour after stimulation. Lysates were collected by washing the cells twice with PBS and harvesting in lysis buffer 6 (Part no. 895561: R&D Systems antibody array ARY002). The cells were resuspended by pipetting, transferred to a 1.5 mL microfuge tube and mixed by rotation at 4° C. for 30 minutes. Lysates were then centrifuged at 14000×g for five minutes and the supernatant was transferred to a clean tube. Protein concentration was determined by the bicinchoninic acid (BCA) protein assay (Pierce, Rockford, Ill.).

### Human Phospho-MAPK Antibody Array

The human phospho-MAPK antibody array was screened against BxPC-3 cell lysates according to the protocol described by the manufacturer (Fourth Revision, May 2006, R&D Systems antibody array ARY002). Briefly, each human phospho-MAPK profiler membrane was prepared by incubating in 1.5 mL of array buffer 1 (Part no. 895477: R&D Systems antibody array ARY002) for 1 hour on a rocking platform shaker. For each treatment, 150 micrograms of total protein was diluted with lysis buffer 6 to give a final volume of 250 microliters and mixed with 1.25 mL of array buffer 1. This mixture was added to the prepared profiler membranes and incubated at 4° C. overnight on a rocking platform shaker. Each membrane was then washed 3 times in 1× wash buffer (diluted in purified distilled water from a 25× stock, (Part no. 895003: R&D Systems antibody array ARY002)) and incubated for 2 hours with 1.5 mL of anti-phospho-MAPK detection antibody cocktail (containing biotinylated phospho-specific antibodies) (Part no. 893051: R&D Systems antibody array ARY002) prepared in 1× array buffer ⅔ (5× array buffer 2, Part no. 895478: R&D Systems antibody array ARY002; array buffer 3, Part no. 895008: R&D Systems antibody array ARY002). The membranes were washed 3 times in 1× wash buffer and incubated for 30 minutes with 1.5 mL of Streptavidin-HRP (Part no. 890803: R&D Systems antibody array ARY002) diluted 1:2000 in 1× array buffer ⅔. The membranes were washed 3 times in 1× wash buffer and exposed to ECL plus Western detection reagents (GE Healthcare, Life Sciences, Piscataway, N.J.) for developing. Membranes were exposed to chemiluminescent film (Kodak, Rochester, N.Y.) and developed using an X-ray medical processor. Phospho-MAPK array data on developed X-ray films were quantitated by scanning the film on a transmission-mode scanner and analyzing the array image file using Image J analysis software (Image J 1.37v, NIH). For each kinase, the average pixel density for corresponding duplicate spots was calculated and subtracted from background signal using the pixel density of a clear area on the membrane. The average normalized pixel density of AR47A6.4.2-treated samples was divided by the average normalized pixel density of 8A3B.6-treated samples for each corresponding phospho-protein target to obtain a ratio of relative change. The percent reduction of phospho-protein signal was determined by subtracting the ratio of relative change from 1 and multiplying by 100.

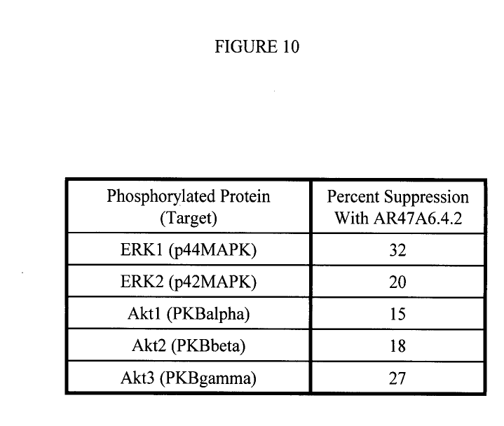

The result from phospho-MAPK array membranes incubated with AR47A6.4.2 or 8A3B.6 is shown in FIG. 10. Compared with 8A3B.6, AR47A6.4.2 suppressed the phosphorylation of p42/p44 MAPK/extracellular signal-regulated kinases (ERK) (ERK1 (32 percent) and ERK2 (20 percent), Akt/protein kinase B (PKB) (Akt1/PKBalpha (15 percent), Akt2/PKBbeta (18 percent) and Akt3/PKBgamma (27 percent)) in BxPC-3 cells stimulated with serum and supplements. These kinases are involved in intracellular signaling pathways that can affect cell proliferation, growth and survival. That AR47A6.4.2 can reduce the phosphorylation of these kinases upon stimulation by serum and supplements suggest that AR47A6.4.2 may block cell growth and survival of cancer cells through these kinases and their related intracellular signaling pathways.

### EXAMPLE 5

### TranSignal™ Angiogenesis Antibody Array of Conditioned Media

To determine whether AR47A6.4.2 treatment can affect secretion of angiogenic factors, conditioned media from cells treated with AR47A6.4.2 were screened using an anangiogenesis array (MA6310, Panomics Inc., Redwood City, Calif.).

### Treatment and Preparation of Cells

As disclosed in Ser. No. 11/709,676, in vivo efficacy of AR47A6.4.2 was demonstrated in a pancreatic cancer xenograft model using BxPC-3 cells grown in severe combined immunodeficient (SCID) mice. Accordingly, screening for secretion of angiogenic factors was performed using the BxPC-3 cell line. BxPC-3 cells were grown to near confluence, washed with phosphate buffered saline (PBS) and then replenished with 2 mL of serum-deficient media. AR47A6.4.2 (20 micrograms/mL) or 8A3B.6 (isotype control; IgG2a) (20 micrograms/mL) was added to the cells and allowed to bind for 20 minutes at 4° C. The cells were placed in an incubator at 37° C. for 24 hours. After 24 hours, the conditioned media from each culture was collected and centrifuged at 1200 revolutions per minute (rpm) for 5 minutes to remove cells or cell debris.

### TranSignal™ Angiogenesis Antibody Array

TranSignal™ angiogenesis antibody arrays were screened with BxPC-3 cell conditioned media according to the protocol described by the manufacturer (Released Oct. 7, 2003, Revised Aug. 3, 2005; MA6310, Panomics Inc., Redwood City, Calif.). Briefly, each TranSignal™ angiogenesis antibody array membrane was prepared by incubating in 3 mL of 1× Blocking Buffer (MA6310, Panomics Inc., Redwood City, Calif.) for 1 hour at room temperature on a rocking platform shaker. The membranes were then washed twice with 4 mL of 1× Wash Buffer II (20× Wash Buffer II diluted to 1× with distilled water (dH₂O), MA6310, Panomics Inc., Redwood City, Calif.). After washing, the entire conditioned media collected (2 mL) was added to a membrane and incubated overnight at 4° C. on a rocking platform shaker. The membranes were then washed 3× using 4 mL of 1× Wash Buffer I (20× wash Buffer diluted to 1× in dH₂O, MA6310, Panomics Inc., Redwood City, Calif.). This was followed by 3 washes with 4 mL of 1× Wash Buffer II (MA6310, Panomics Inc., Redwood City, Calif.). The membranes were then incubated for 1 hour in 1.5 mL of Biotin-Conjugated Anti-Angiogenesis Mix (MA6310, Panomics Inc., Redwood City, Calif.) on a rocking platform shaker, washed 3× using 4 mL of 1× Wash Buffer I (MA6310, Panomics Inc., Redwood City, Calif.) followed by 3 washes with 4 mL of 1× Wash Buffer II (MA6310, Panomics Inc., Redwood City, Calif.). Strepavidin-HRP, diluted 1:1000 in 1× Wash Buffer II, was added to the membranes and incubated for 1 hour at room temperature, washed again 3× using 4 mL of 1× Wash Bufffer I (MA6310, Panomics Inc., Redwood City, Calif.) followed by 3 washes with 4 mL of 1× Wash Buffer II (MA6310, Panomics Inc., Redwood City, Calif.) and developed using Hyperfilm ECL reagent (RPN3114K, GE Healthcare, Life Sciences, Piscataway, N.J.). The membranes were exposed to chemiluminescent film (Kodak, Rochester, N.Y.) and developed using an X-ray medical processor. Angiogenesis array data on developed X-ray films were quantitated by scanning the film on a transmission-mode scanner and analyzing the array image file using Image J analysis software (Image J 1.37v, NIH). For each secreted factor, the average pixel density for corresponding duplicate spots was calculated and subtracted from background signal using the pixel density of a clear area on the membrane. The average normalized pixel density of AR47A6.4.2-treated samples was divided by the average normalized pixel density of 8A3B.6-treated samples for each corresponding target to obtain a ratio of relative change. The percent reduction of signal was determined by subtracting the ratio of relative change from 1 and multiplying by 100.

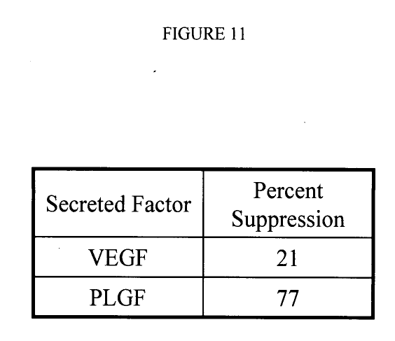

The results from TranSignal™ angiogenesis antibody array membranes incubated with AR47A6.4.2 or 8A3B.6 are shown in FIG. 11. Compared with 8A3B.6, AR47A6.4.2 suppressed the secretion of the potent angiogenic factors vascular endothelial growth factor (VEGF) and placental growth factor (PLGF). This observation suggests that treatment of the BxPC-3 pancreatic cancer cell line with AR47A6.4.2 may result in the inhibition of tumor growth and survival of the cancer cells by reducing the secretion of factors by the cells that promote blood vessel growth in solid tumors. This finding demonstrates a possible mechanism of action for AR47A6.4.2.

### EXAMPLE 6

### Demonstration of In Vitro Complement-Dependent Cytotoxicity (CDC) Activity of the Anti-TROP-2 Antibody AR47A6.4.2

Therapeutic efficacy of murine AR47A6.4.2 has been previously demonstrated in xenograft tumor models of human pancreatic cancer (as disclosed in Ser. No. 11/709,676 and in Example 2 above). In order to elucidate its mechanisms of action, AR47A6.4.2 was evaluated in vitro for CDC activity on two pancreatic cancer cell lines, PL45 and BxPC-3. Established monolayers of PL45 and BxPC-3 cells, two days post plating, were treated with antibody (2, 0.2 and 0.02 micrograms/mL) and allowed to bind for one hour (37° C.; 4 percent CO₂). Rabbit complement was then added to yield a final concentration of 10 percent (v/v) and cells were allowed to incubate for an additional 3 hours at 37° C., 4 percent CO₂. CDC activity was evaluated by measuring the residual lactate dehydrogenase present in uncompromised cells using the Cytotox 96™ kit (Promega Corporation, Madison, Wis., USA). Each test antibody was evaluated in triplicate and the results were expressed as percent cytotoxicity, as compared to rabbit complement only treated wells, using the following equation: percent Cytotoxicity=100−[Test Antibody_{(492 nm)}−Background_{(492 nm)}]/Complement Only_{(492 nm)}−Background_{(492 nm)}]*100.

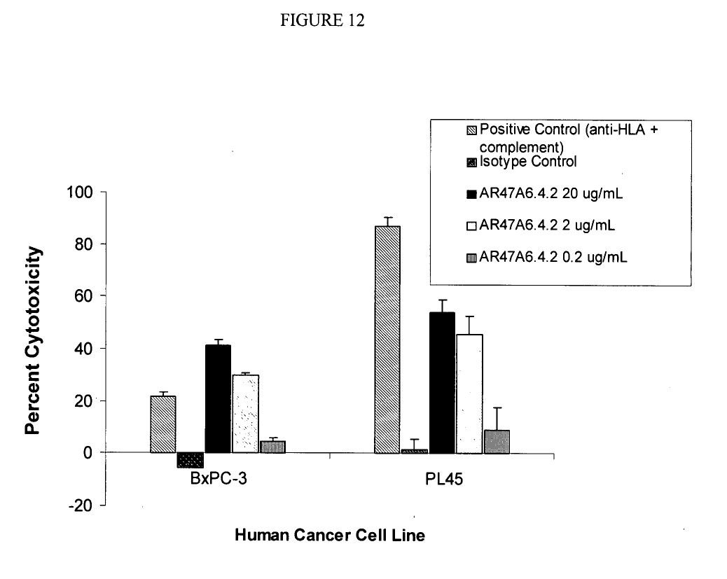

The results from this experiment (FIG. 12) demonstrate that the anti-TROP-2 antibody AR47A6.4.2 was capable of recruiting rabbit complement in a dose-dependent manner in both pancreatic cancer target cell lines (PL45 and BxPC-3). CDC activity was not observed in these cell lines when treated with isotype-matched control at the highest concentration (20 micrograms/mL). This data demonstrates that AR47A6.4.2 is capable of complement recruitment in vitro and may be one of the mechanisms by which this antibody is exerting its effects in vivo.

### EXAMPLE 7

### Epitope Mapping

Epitope mapping experiments were carried out in order to determine the region(s) of the TROP-2 molecule that are recognized by AR47A6.4.2. Overlapping 15-mer peptides were synthesized based on the amino acid sequence of TROP-2 using standard Fmoc-chemistry and deprotected using trifluoric acid with scavengers. Additionally, up to 30-mer double-looped, triple-looped and sheet-like peptides were synthesized on chemical scaffolds in order to reconstruct discontinuous epitopes of the TROP-2 molecule, using Chemically Linked Peptides on Scaffolds (CLIPS) technology. The looped peptides were synthesized containing a dicysteine, which was cyclized by treating with alpha,alpha′-dibromoxylene and the size of the loop was varied by introducing cysteine residues at variable spacing. If other cysteines besides the newly introduced cysteines were present, they were replaced by an alanine. The side-chains of the multiple cysteines in the peptides were coupled to CLIPS templates by reacting onto credit-card format polypropylene PEPSCAN cards (455 peptide formats/card) with an 0.5 mM solution of CLIPS template such as 1,3-bis(bromomethyl)benzene in ammonium bicarbonate (20 mM, pH 7.9)/acetonitrile (1:1 (v/v)). The cards were gently shaken in the solution for 30 to 60 minutes while completely covered in solution. Finally, the cards were washed extensively with excess H₂O and sonicated in disrupt-buffer containing 1 percent SDS/0.1 percent beta-mercaptoethanol in PBS (pH 7.2) at 70° C. for 30 minutes, followed by sonication in H₂O for another 45 minutes. In total, 3579 different peptides were synthesized. The binding of antibody to each peptide was tested in a PEPSCAN-based ELISA. The 455-well credit card format polypropylene cards containing the covalently linked peptides were incubated with primary antibody solution consisting of 10 micrograms/mL of AR47A6.4.2 diluted in blocking solution (5% horse-serum (v/v), 5% ovalbumin (w/v) and 1% Tween 80 in PBS) overnight. After washing with PBS containing 1% Tween 80, the peptides were incubated with a 1/1000 dilution of rabbit anti-mouse antibody peroxidase in blocking solution (5 percent horse-serum (v/v), 5 percent ovalbumin (w/v) and Tween 80 in PBS) for one hour at 25° C. After washing with PBS containing 1 percent Tween 80, the peroxidase substrate 2,2′-azino-di-3-ethylbenzthiazoline sulfonate (ABTS) and 2 microliters of 3 percent H₂O₂ were added. After one hour, the color development was measured. The color development was quantified on a logarithmic scale of 0 to 4000 with a charge coupled device (CCD)-camera and an image processing system.

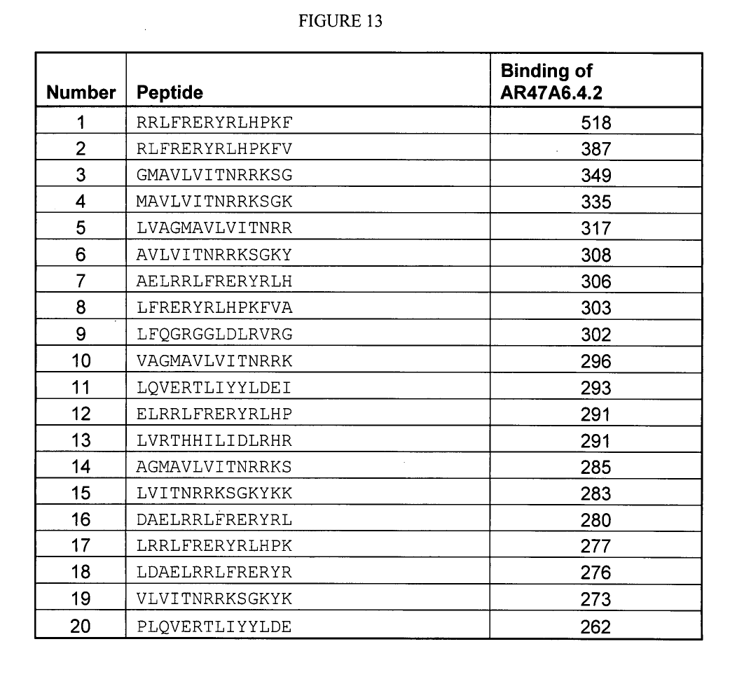

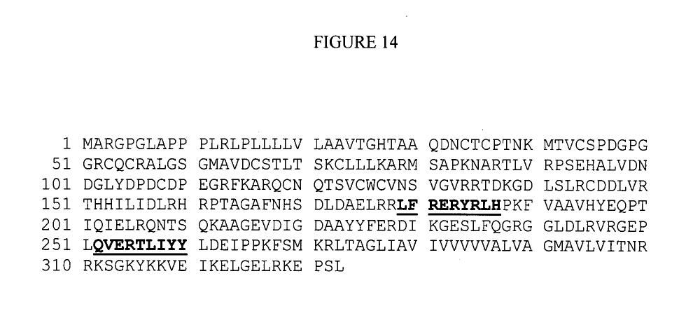

The twenty peptides (out of 3579) to which AR47A6.4.2 bound most strongly are listed in FIG. 13. Two amino acid hotspots were identified by analyzing the composition of the peptides to which AR47A6.4.2 bound. The hotspot amino acid sequence LFRERYRLH is present in peptide numbers 1, 2, 7, 8, 12, 16, 17 and 18 and the hotspot amino acid sequence QVERTLIYY is present in peptide numbers 11 and 20. Peptides 3-6, 10, 14, 15 and 19 most likely represent an epitope mimic, as the sequence of these peptides falls within the intracellular portion of the TROP-2 molecule. Overall these results indicate that AR47A6.4.2 recognizes a discontinuous epitope consisting of sequences around LFRERYRLH and QVERTLIYY. The position of these amino acid sequences within the entire TROP-2 molecule amino acid sequence is presented in FIG. 14.

### EXAMPLE 8

### Humanization of AR47A6.4.2

Recombinant DNA techniques were performed using methods well known in the art and, as appropriate, supplier instructions for use of enzymes used in these methods. Detailed laboratory methods are also described below.

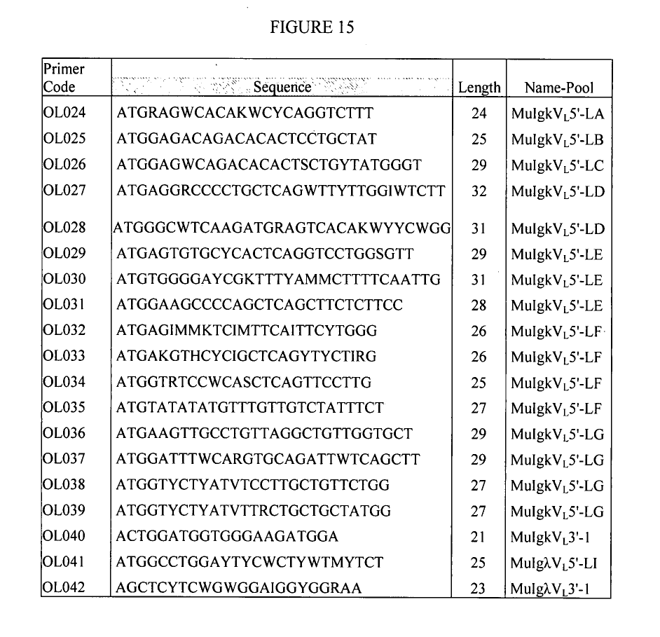

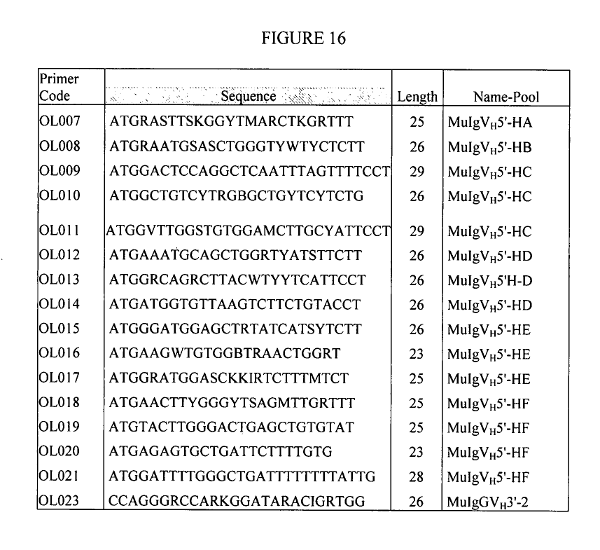

mRNA was extracted from the hybridoma AR47A6.4.2 cells using a Poly A Tract System 1000 mRNA extraction kit: (Promega Corp., Madison, Wis.) according to manufacturer's instructions. mRNA was reverse transcribed as follows: For the kappa light chain, 5.0 microliters of mRNA was mixed with 1.0 microliter of 20 μmol/microliter MuIgGκV_{L}-3′ primer OL040 (FIG. 15) and 5.5 microliters nuclease free water (Promega Corp., Madison, Wis.). For the lambda light chain, 5.0 microliters of mRNA was mixed with 1.0 microliter of 20 μmol/microliter MuIgGλV_{L}-3′ primer OL042 (FIG. 15) and 5.5 microliters nuclease free water (Promega Corp., Madison, Wis.). For the gamma heavy chain, 5 microliters of mRNA was mixed with 1.0 microliter of 20 μmol/microliter MuIgGV_{H}-3′ primer OL023 (FIG. 16) and 5.5 microliter nuclease free water (Promega Corp., Madison, Wis.). All three reaction mixes were placed in the pre-heated block of the thermal cycler set at 70° C. for 5 minutes. These were chilled on ice for 5 minutes before adding to each 4.0 microliters ImPromII 5× reaction buffer (Promega Corp., Madison, Wis.), 0.5 microliters RNasin ribonuclease inhibitor (Promega Corp., Madison, Wis.), 2.0 microliters 25 mM MgCl₂ (Promega Corp., Madison, Wis.), 1.0 microliter 10 mM dNTP mix (Invitrogen, Paisley, UK) and 1.0 microliter Improm II reverse transcriptase (Promega Corp., Madison, Wis.). The reaction mixes were incubated at room temperature for 5 minutes before being transferred to a pre-heated PCR block set at 42° C. for 1 hour. After this time the reverse transcriptase was heat inactivated by incubating at 70° C. in a PCR block for fifteen minutes.

Heavy and light chain sequences were amplified from cDNA as follows: A PCR master mix was prepared by adding 37.5 microliters 10× Hi-Fi Expand PCR buffer: (Roche, Mannheim, Germany), 7.5 microliters 10 mM dNTP mix (Invitrogen, Paisley, UK) and 3.75 microliters Hi-Fi Expand DNA polymerase (Roche, Mannheim, Germany) to 273.75 microliters nuclease free water. This master mix was dispensed in 21.5 microliter aliquots into 15 thin walled PCR reaction tubes on ice. Into six of these tubes was added 2.5 microliters of MuIgV_{H}-3′ reverse transcription reaction mix and 1.0 microliter of heavy chain 5′ primer pools HA to HF (see FIG. 16 for primer sequences and primer pool constituents). To another seven tubes was added 2.5 microliters of MuIgKVL-3′ reverse transcription reaction and 1.0 microliter of light chain 5′ primer pools LA to LG (FIG. 15). Into the final tube was added 2.5 microliters of MuIgKVL-3′ reverse transcription reaction and 1.0 microliter of lambda light chain primer MuIgKVL5′-L1. Reactions were placed in the block of the thermal cycler and heated to 95° C. for 2 minutes. The polymerase chain reaction (PCR) reaction was performed for 40 cycles of 94° C. for 30 seconds, 55° C. for 1 minute and 72° C. for 30 seconds. Finally the PCR products were heated at 72° C. for 5 minutes, and then held at 4° C.

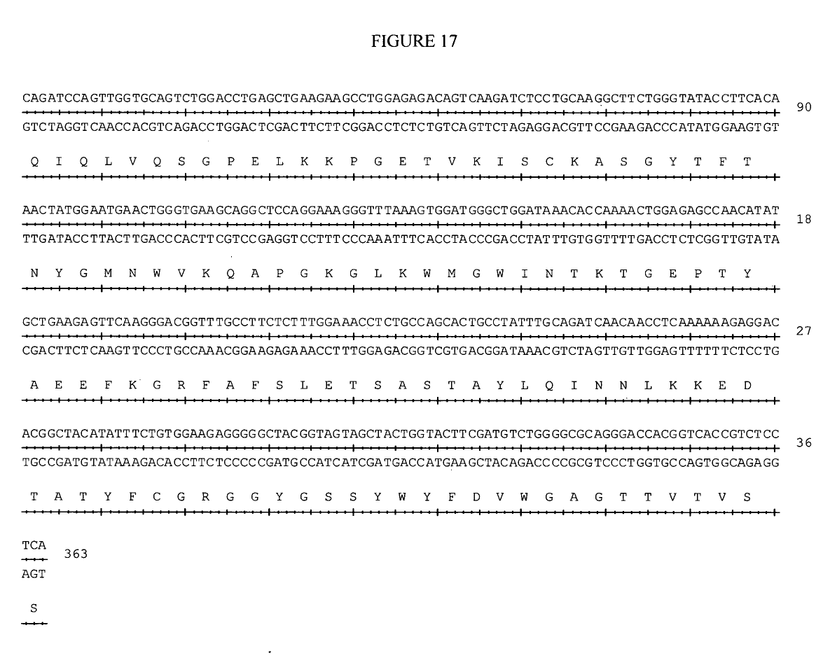

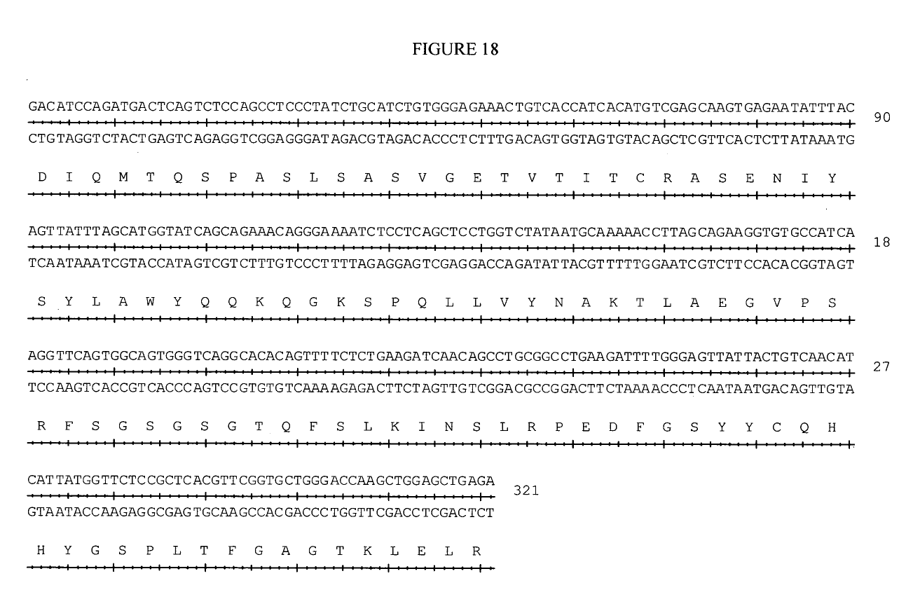

Amplification products were cloned into pGEM-T easy vector using the pGEM-T easy Vector System I (Promega Corp., Madison, Wis.) kit and sequenced. The resultant VH and VL sequences are shown in FIGS. 17 and 18 respectively.

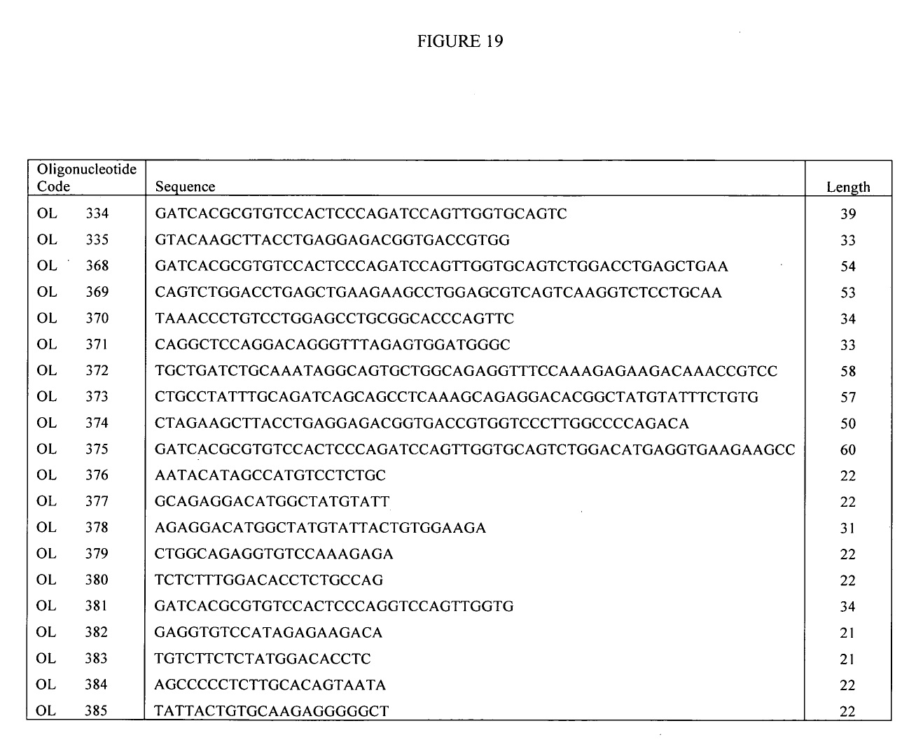

For generation of a chimeric antibody, VH region genes were amplified by PCR using the primers OL334 and OL335 (FIG. 19); these were designed in order to engineer in a 5′ MluI and a 3′ HindIII restriction enzyme site using plasmid DNA from one of the cDNA clones as a template. Into a 0.5 mL PCR tube was added 5 microliters 10× Hi-Fi Expand PCR buffer: (Roche, Mannheim, Germany), 1.0 microliter 10 mM dNTP mix (Invitrogen, Paisley, UK), 0.5 microliters of Primer OL330, 0.5 microliters of primer OL331, 1.0 microliter template DNA and 0.5 microliters Hi-Fi Expand DNA polymerase (Roche, Mannheim, Germany) to 41.5 microliters nuclease free water.

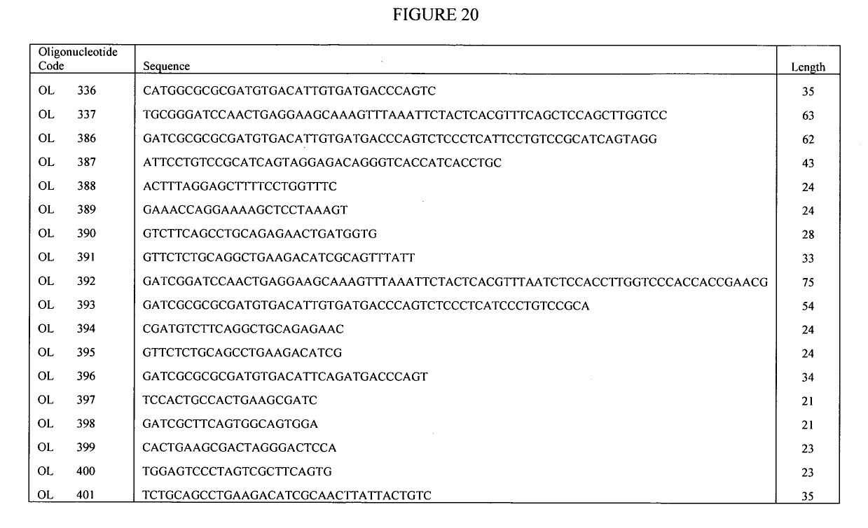

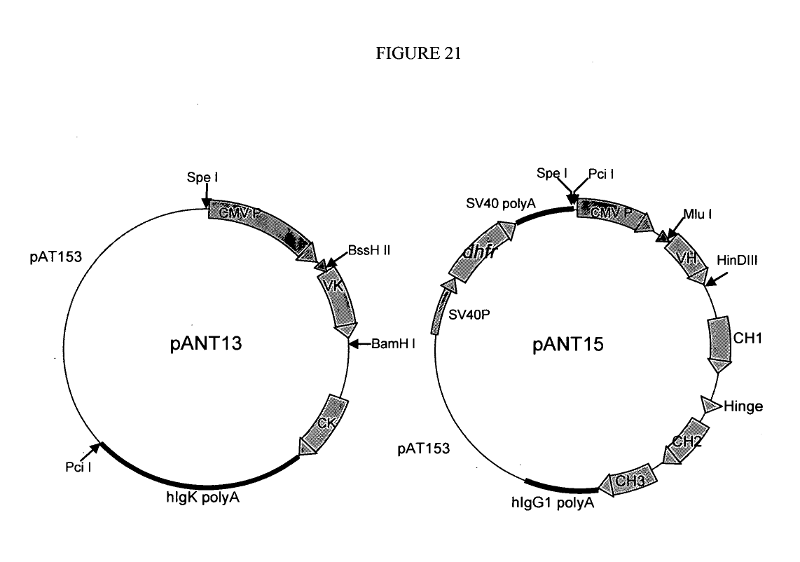

VL regions were amplified in a similar method using the oligonucleotides OL336 and OL337 (FIG. 20) to engineer in BssHII and BamHI restriction enzyme sites. Reactions were placed in the block of the thermal cycler and heated to 95° C. for 2 minutes. The polymerase chain reaction (PCR) reaction was performed for 30 cycles of 94° C. for 30 seconds, 55° C. for 1 minute and 72° C. for 30 seconds. Finally the PCR products were heated at 72° C. for 5 minutes, and then held at 4° C. VH and VL region PCR products were then cloned into the vectors pANT15 and pANT13 respectively (FIG. 21) at the MluI/HindIII and BssHII/BamHI sites respectively. Both pANT15 and pANT13 are pAT153-based plasmids containing a human Ig expression cassette. The heavy chain cassette in pANT15 consists of a human genomic IgG1 constant region gene driven by the hCMVie promoter, with a downstream human IgG polyA region. pANT15 also contains a hamster dhfr gene driven by the SV40 promoter with a downstream SV40 polyA region. The light chain cassette of pANT13 is comprised of the genomic human kappa constant region driven by the hCMVie promoter with downstream light chain polyA region. Cloning sites between a human Ig leader sequence and the constant regions allow for the insertion of the variable region genes.

NS0 cells (ECACC 85110503, Porton, UK) were co-transfected with these two plasmids via electroporation and selected in DMEM (Invitrogen, Paisley, UK) plus 5 percent FBS (Ultra low IgG Cat No. 16250-078 Invitrogen, Paisley, UK) plus Penicillin/Streptomycin (Invitrogen, Paisley, UK) plus 100 nM Methotrexate (Sigma, Poole, UK). Methotrexate resistant colonies were isolated and antibody was purified by Protein A affinity chromatography using a 1 mL HiTrap MabSelect SuRe column (GE Healthcare, Amersham, UK) following the manufacturers recommended conditions.

The chimeric antibody was tested in an ELISA-based competition assay using AR47A6.4.2 mouse antibody, biotinylated using Biotintag micro biotinylation kit (Sigma, Poole, UK). Biotinylated mouse AR47A6.4.2 was used to bind OVCAR-3 cells in the presence of varying concentrations of competing antibody. OVCAR-3 cells were cultured to near confluence in tissue culture treated, flat bottomed, 96 well plates and then fixed. Biotinylated mouse AR47A6.4.2 antibody was diluted to 1 microgram/mL and mixed with an equal volume of competing antibody at concentrations ranging from 0-5 micrograms/mL. 100 microliters of the antibody mixes were transferred into the wells of the OVCAR-3 coated plate and incubated at room temperature for 1 hour. The plate was washed, and bound biotinylated mouse AR47A6.4.2 was detected by adding a strepavidin-HRP conjugate (Sigma, Poole, UK) (diluted at 1:500) and OPD substrate (Sigma, Poole, UK). The assay was developed in the dark for 5 minutes before being stopped by the addition of 3 M HCl. The assay plate was then read in a MRX TCII plate reader (Dynex Technologies, Worthing, UK) at an absorbance of 490 nm. The chimeric antibody ((ch)AR47A6.4.2) was shown to be equivalent to the mouse AR47A6.4.2 antibody in competing with biotinylated AR47A6.4.2 antibody for binding to OVCAR-3 cells.

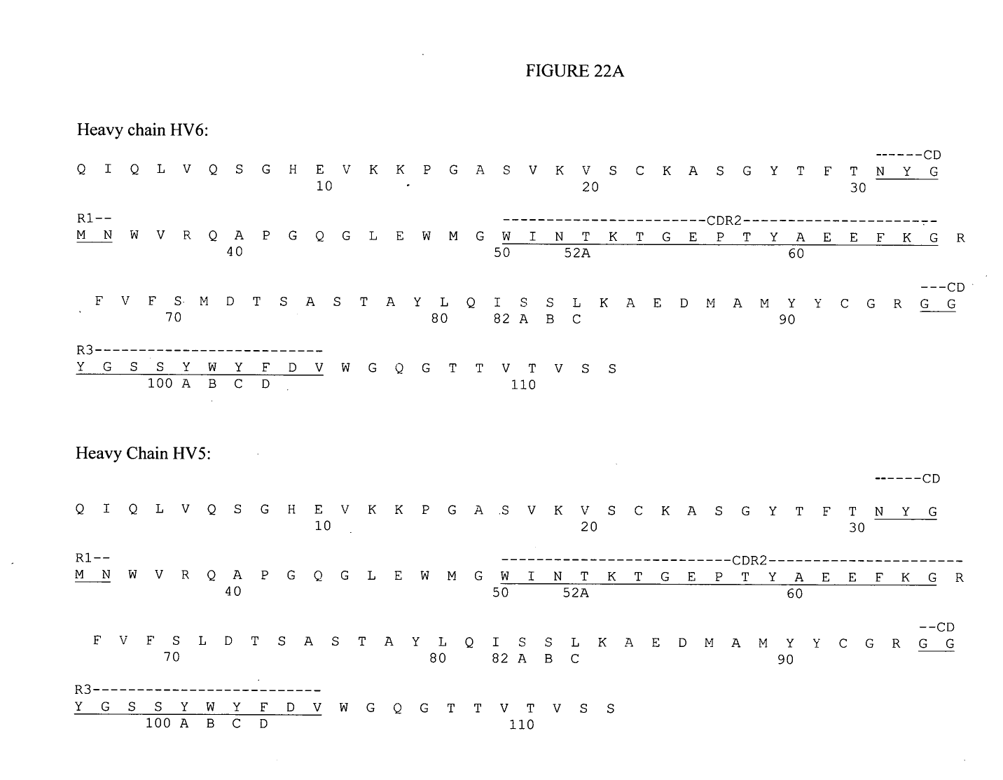

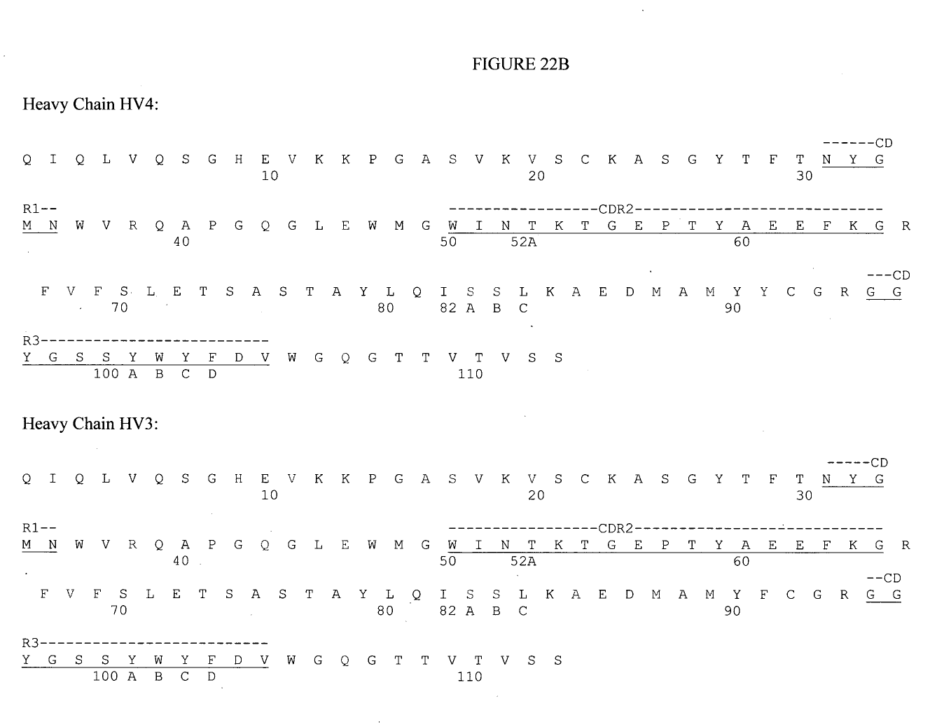

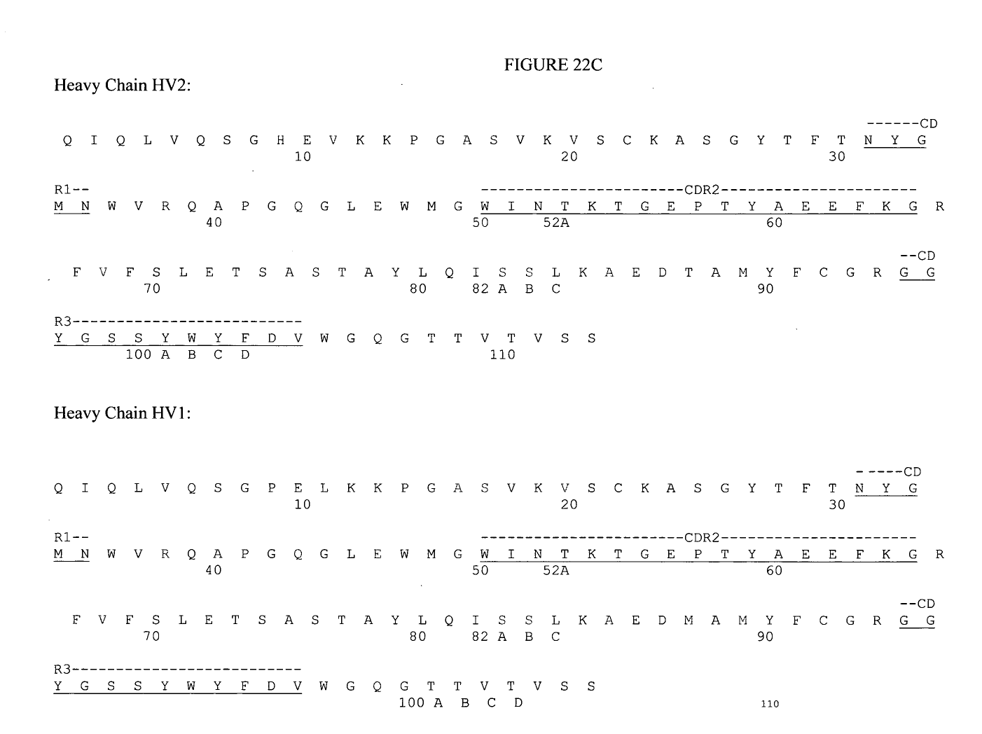

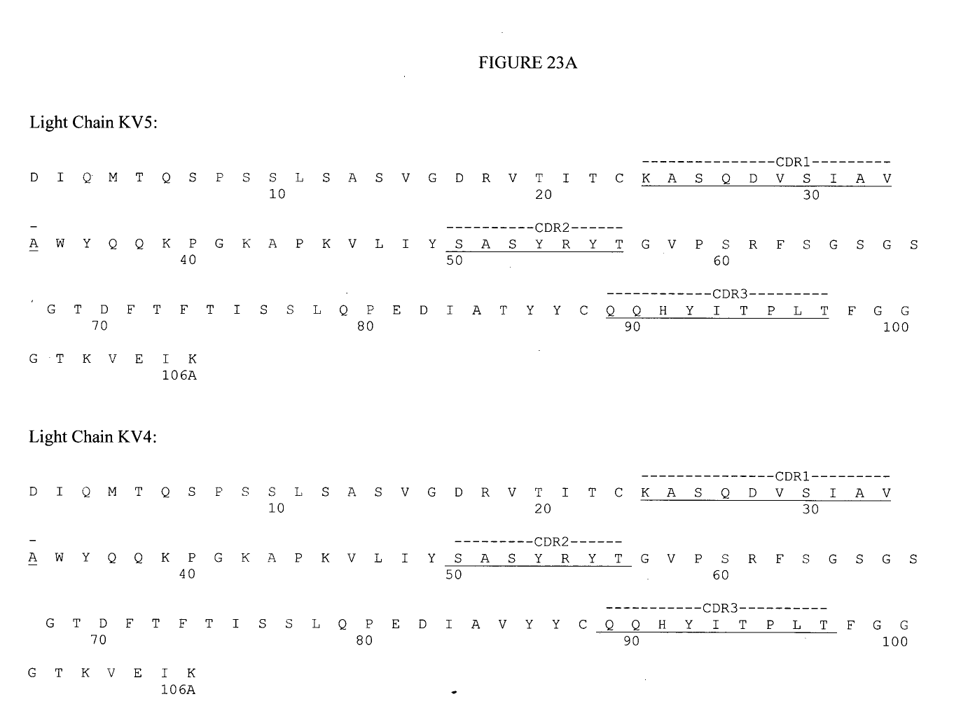

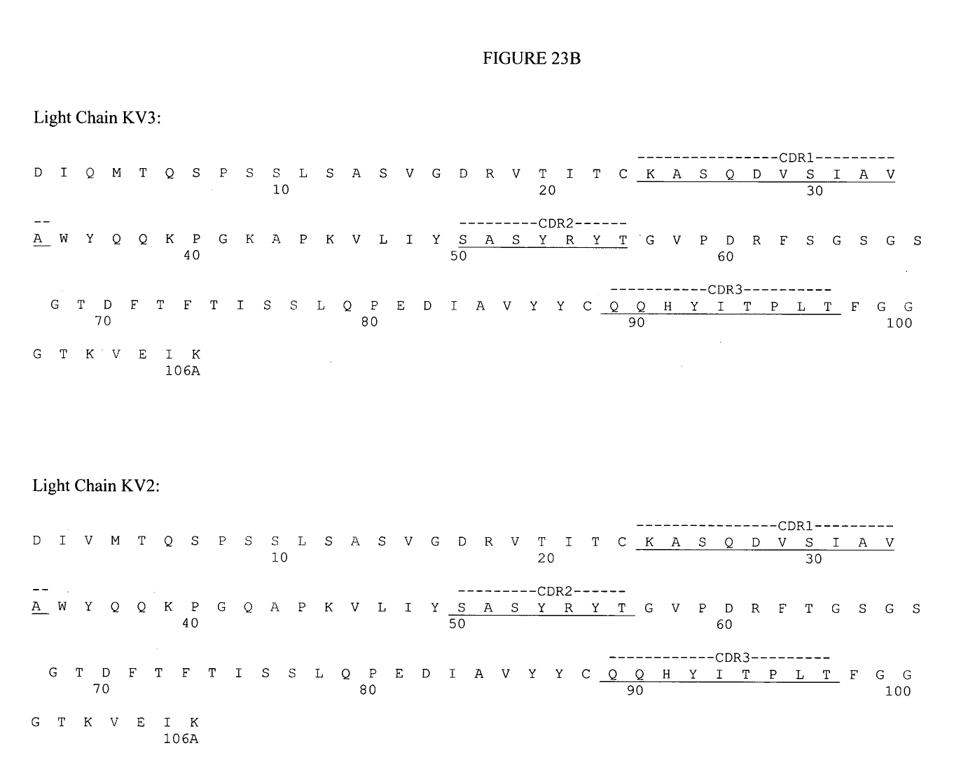

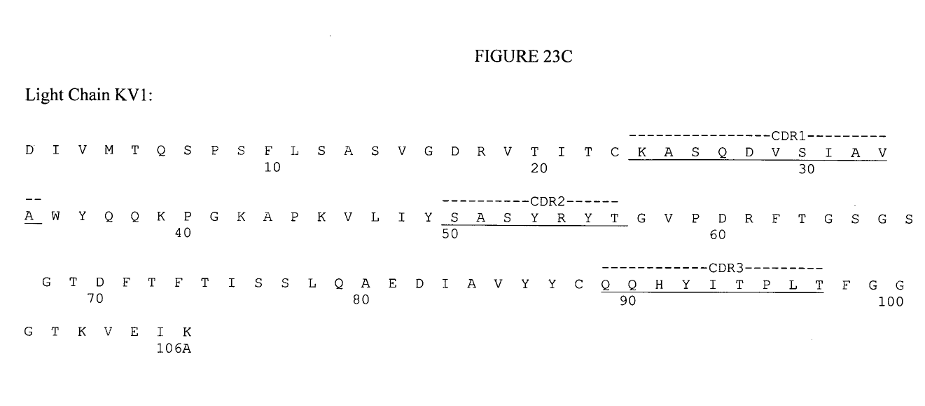

Humanized VH and VL sequences were designed by comparison of mouse AR47A6.4.2 sequences and homologous human VH and VL sequences. Sequences of the VH variants are given in FIG. 22 and of the VL variants in FIG. 23. Humanized V region genes were constructed using the mouse AR47A6.4.2 VH and VL templates for PCR using long overlapping oligonucleotides to introduce amino acids from homologous human VH and VL sequences. Oligonucleotides used for the generation of variant humanized VH and VL sequences are shown in FIGS. 19 and 20 respectively. Variant genes were cloned directly into the expression vectors pSVgpt and pSVhyg as detailed in US2004260069 (Hellendoom, Carr and Baker).

All combinations of variant humanized heavy and light chains (including the chimeric constructs) were transiently transfected into CHO-K1 cells (ECACC 85051005, Porton, UK) and supernatants harvested after 48 hours. The supernatants were quantified for antibody expression in a IgG Fc/Kappa ELISA using purified human IgG1/Kappa (Sigma, Poole, UK) as standards. Immunosorb 96 well plates (Nalge nunc, Hereford, UK) were coated with mouse anti-human IgG Fc-specific antibody (16260 Sigma, Poole, UK) diluted at 1:1500 in 1×PBS (pH 7.4) at 37° C. for 1 hour. Plates were washed three times in PBS+0.05 percent Tween 20 before adding samples and standards, diluted in 2 percent BSA/PBS. Plates were incubated at room temperature for 1 hour before washing three times in PBS/Tween and adding 100 microliters/well of detecting antibody goat anti-human kappa light chain peroxidase conjugate (A7164 Sigma, Poole, UK) diluted 1:1000 in 2 percent BSA/PBS. Plates were incubated at room temperature for 1 hour before washing five times with PBS/Tween. Bound antibody was detected using OPD substrate (Sigma, Poole, UK). The assay was developed in the dark for 5 minutes before being stopped by the addition of 3 M HCl. The assay plate was then read in a MRX TCII plate reader (Dynex Technologies, Worthing, UK) at 490 nm.

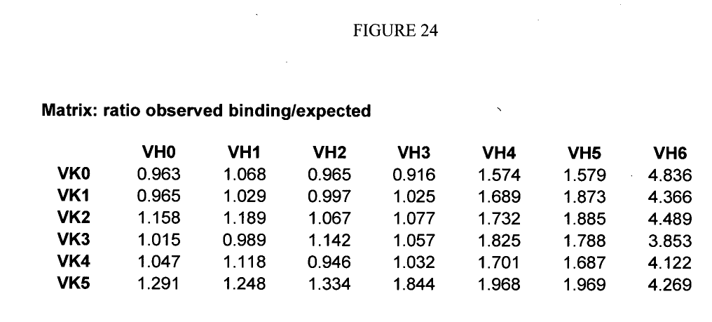

Binding of the humanized variants was assayed in the competition binding ELISA described above. A standard curve was generated with varying concentrations (156.25 ng/mL to 5 micrograms/mL) of purified chimeric antibody ((ch)AR47A6.4.2) competing for binding with mouse AR47A6.4.2 to fixed OVCAR-3 cells on a 96-well microtitre plate. Binding of mouse AR47A6.4.2 to OVCAR-3 cells was detected with goat anti-mouse IgG:HRP conjugate (A2179 Sigma, Poole, UK) and developed using TMB substrate (Sigma, Poole, UK) Using the chimeric standard curve, the percentage inhibition expected at the concentrations tested was calculated for each variant and compared to that actually observed. The results were then normalized by dividing the observed inhibition of the test sample by the expected inhibition for each of the various heavy/light chain combinations. Thus a sample with an observed/expected ratio=1.0 has the same binding affinity as the chimeric antibody whereas a value >1.0 has reduced binding to TROP-2 and a sample with a ratio <1.0 has improved binding to TROP-2. The results are shown in FIG. 24.

Combinations of VH and VL genes were cloned into the dual vector pANT18 (pANT 18 vector is based on the plasmid pANT15 described previously, with the light chain cassette from pANT13 cloned into the SpeI/PciI restriction enzyme sites) and transfected into CHO/dhfr-cells (ECACC, 94060607) by electroporation and selected in media (high glucose DMEM with L-glutamine and Na pyruvate (Invitrogen, Paisley UK) plus 5 percent dialysed FBS (Cat No. 26400-044 Invitrogen, Paisley, UK), Proline (Sigma, Poole, UK) and Penicillin/Streptomycin (Invitrogen, Paisley, UK) depleted of Hypoxanthine and Thymidine. Antibodies were purified by Protein A affinity chromatography as above. The purified antibodies were filter sterilized before storing (in PBS pH 7.4) at +4° C. The concentrations of the antibodies were calculated by human IgG1/kappa capture ELISA as above.

Four of the purified antibody samples were tested for binding to OVCAR-3 cells expressing human TROP-2 via a competition ELISA as above. Varying concentrations of each antibody (156 ng/mL to 5 micrograms/mL) were mixed with purified mouse AR47A6.4.2 and added to microtiter plates coated with fixed OVCAR-3 cells. Binding of mouse AR47A6.4.2 was detected with goat anti-mouse IgG (Fc):HRP conjugate as above. Absorbance at 450 nm was measured on a plate reader and this was plotted against the test antibody concentration. The concentration of selected variants required to inhibit mouse AR47A6.4.2 binding to OVCAR-3 cells by 50 percent (IC₅₀) was calculated and compared to the chimeric antibody.

The IC₅₀ for the four variant humanized antibodies and the chimeric antibody are as follows:

(ch)AR47A6.4.2=1.71 micrograms/mL

(hu)AR47A6.4.2 variant HV2/KV3=2.24 micrograms/mL

(hu)AR47A6.4.2 variant HV2/KV4=3.04 micrograms/nL

(hu)AR47A6.4.2 variant HV3/KV3=2.04 micrograms/mL

(hu)AR47A6.4.2 variant HV3/KV4=1.02 micrograms/mL

### EXAMPLE 9

### Determination of the Binding Affinity of AR47A6.4.2 and (hu)AR47A.6.4.2 to rhTROP-2

The binding affinity of AR47A6.4.2, (hu)AR47A6.4.2 variant HV2/KV3, (hu)AR47A6.4.2 variant HV2/KV4, (hu)AR47A6.4.2 variant HV3/KV3 and (hu)AR47A6.4.2 variant HV3/KV4 was compared by the determination of the respective dissociation constants (K_{D}) subsequent to binding to recombinant human TROP-2 (rhTrop-2).

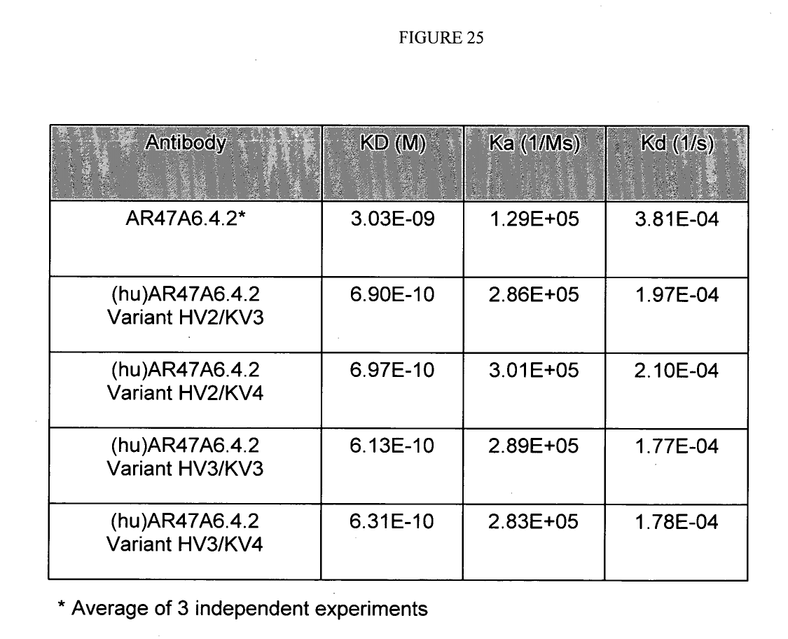

An anti-polyHistidine monoclonal antibody (R&D Systems, Minneapolis, Minn., USA) was immobilized using the standard amine coupling procedure. The surface of a CM5 sensor chip (GE Healthcare, Piscataway, N.J. USA formerly Biacore) was activated by injection of 104 microliters of a 1:1 mixture of 0.4M EDC and 0.1 M NHS (flow rate 10 microliters/minute). The anti-polyHistidine antibody was injected at a concentration of 20 micrograms/mL (diluted in 10 mM sodium acetate pH 5.5) to reach approximately 2000 RU. Finally, 119 microliters of 1.0 M ethanolamine-HCL pH 8.5 was injected over the surface to block any unoccupied activated sites on the sensor chip surface. HIS-tagged rhTROP-2 (R&D Systems, Minneapolis, Minn., USA) was injected at 1 microgram/mL and captured by the HIS tag on the chip surface, followed by injection of AR47A6.4.2, (hu)AR47A6.4.2 variant HV2/KV3, (hu)AR47A6.4.2 variant HV2/KV4, (hu)AR47A6.4.2 variant HV3/KV3 or (hu)AR47A6.4.2 variant HV3/KV4. Regeneration of the sensor chip surface for subsequent injections was accomplished by injection of 10 mM Glycine-HCl pH 2.0 for 60 seconds at a flow rate of 50 microliters/minute. Antibodies were diluted in running buffer (HBS-EP+, GE Healthcare, Piscataway, N.J. USA formerly Biacore) and serially injected at concentrations ranging from 0.67 to 333 nM, and the surface was regenerated between each cycle. As a control, each antibody concentration was also injected over a reference surface, which had immobilized anti-polyHistidine antibody but did not have captured rhTROP-2 on the surface. Using Biacore T100 Evaluation Software Version 1.1, kinetic analysis was performed on the obtained sensograms using a simple 1:1 interaction model. The association and dissociation rate constants measured were used to calculate the KD of the antibodies. The experiments were conducted using a Biacore T100 system (GE Healthcare, Piscataway, N.J. USA formerly Biacore). The results of these experiments yielded values of 3.03 nM for murine AR47A6.4.2 while all four (hu)AR47A6.4.2 were between 0.613 to 0.697 nM. (FIG. 25), indicating that all of the antibodies are in the nanomolar to subnanomolar range, and that the affinities of the humanized antibodies are higher than that of the parental murine AR47A6.4.2. The association rate constants (Ka) and dissociation rate constants (Kd) were also tabulated (FIG. 25).

### EXAMPLE 10

### Isolation of Competitive Binders

Given an antibody, an individual ordinarily skilled in the art can generate a competitively inhibiting CDMAB, for example a competing antibody, which is one that recognizes the same epitope (Belanger L et al. Clinica Chimica Acta 48:15-18 (1973)). One method entails immunizing with an immunogen that expresses the antigen recognized by the antibody. The sample may include but is not limited to tissues, isolated protein(s) or cell line(s). Resulting hybridomas could be screened using a competition assay, which is one that identifies antibodies that inhibit the binding of the test antibody, such as ELISA, FACS or Western blotting. Another method could make use of phage display antibody libraries and panning for antibodies that recognize at least one epitope of said antigen (Rubinstein J L et al. Anal Biochem 314:294-300 (2003)). In either case, antibodies are selected based on their ability to displace the binding of the original labeled antibody to at least one epitope of its target antigen. Such antibodies would therefore possess the characteristic of recognizing at least one epitope of the antigen as the original antibody.

### EXAMPLE 11

### Cloning of the Variable Regions of the AR47A6.4.2 Monoclonal Antibody

The sequences of the variable regions from the heavy (V_{H}) and light (V_{L}) chains of monoclonal antibody produced by the AR47A6.4.2 hybridoma cell line were previously determined (as disclosed in Ser. No. 11/709,676). To generate chimeric and humanized IgG, the variable light and variable heavy domains can be subcloned into an appropriate vector for expression (as disclosed in Example 8 above).

In another embodiment, AR47A6.4.2 or its de-immunized, chimeric or humanized version is produced by expressing a nucleic acid encoding the antibody in a transgenic animal, such that the antibody is expressed and can be recovered. For example, the antibody can be expressed in a tissue specific manner that facilitates recovery and purification. In one such embodiment, an antibody of the invention is expressed in the mammary gland for secretion during lactation. Transgenic animals include but are not limited to mice, goat and rabbit.

### (i) Monoclonal Antibody

DNA encoding the monoclonal antibody (as disclosed in Ser. No. 11/709,676) is readily isolated and sequenced using conventional procedures (e.g., by using oligonucleotide probes that are capable of binding specifically to genes encoding the heavy and light chains of the monoclonal antibodies). The hybridoma cell serves as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences. Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

### (ii) Humanized Antibody

A humanized antibody has one or more amino acid residues introduced into it from a non-human source. These non-human amino acid residues are often referred to as “import” residues, which are typically taken from an “import” variable domain. Humanization can be performed using the method of Winter and co-workers by substituting rodent CDRs or CDR sequences for the corresponding sequences of a human antibody (Jones et al., Nature 321:522-525 (1986); Riechmann et al., Nature 332:323-327 (1988); Verhoeyen et al., Science 239:1534-1536 (1988); reviewed in Clark, Immunol. Today 21:397-402 (2000)).

A humanized antibody can be prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

(iii) Antibody Fragments

Various techniques have been developed for the production of antibody fragments. These fragments can be produced by recombinant host cells (reviewed in Hudson, Curr. Opin. Immunol. 11:548-557 (1999); Little et al., Immunol. Today 21:364-370 (2000)). For example, Fab′-SH fragments can be directly recovered from E. coli and chemically coupled to form F(ab′)₂ fragments (Carter et al., Biotechnology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture.

### EXAMPLE 12

### A Composition Comprising the Antibody of the Present Invention

The antibody of the present invention can be used as a composition for preventing/treating cancer. The composition for preventing/treating cancer, which comprises the antibody of the present invention, can be administered as they are in the form of liquid preparations, or as pharmaceutical compositions of suitable preparations to human or mammals (e.g., rats, rabbits, sheep, swine, bovine, feline, canine, simian, etc.) orally or parenterally (e.g., intravascularly, intraperitoneally, subcutaneously, etc.). The antibody of the present invention may be administered in itself, or may be administered as an appropriate composition. The composition used for the administration may contain a pharmacologically acceptable carrier with the antibody of the present invention or its salt, a diluent or excipient. Such a composition is provided in the form of pharmaceutical preparations suitable for oral or parenteral administration.

Examples of the composition for parenteral administration are injectable preparations, suppositories, etc. The injectable preparations may include dosage forms such as intravenous, subcutaneous, intracutaneous and intramuscular injections, drip infusions, intraarticular injections, etc. These injectable preparations may be prepared by methods publicly known. For example, the injectable preparations may be prepared by dissolving, suspending or emulsifying the antibody of the present invention or its salt in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mols) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is usually filled in an appropriate ampoule. The suppository used for rectal administration may be prepared by blending the antibody of the present invention or its salt with conventional bases for suppositories. The composition for oral administration includes solid or liquid preparations, specifically, tablets (including dragees and film-coated tablets), pills, granules, powdery preparations, capsules (including soft capsules), syrup, emulsions, suspensions, etc. Such a composition is manufactured by publicly known methods and may contain a vehicle, a diluent or excipient conventionally used in the field of pharmaceutical preparations. Examples of the vehicle or excipient for tablets are lactose, starch, sucrose, magnesium stearate, etc.

Advantageously, the compositions for oral or parenteral use described above are prepared into pharmaceutical preparations with a unit dose suited to fit a dose of the active ingredients. Such unit dose preparations include, for example, tablets, pills, capsules, injections (ampoules), suppositories, etc. The amount of the aforesaid compound contained is generally 5 to 500 mg per dosage unit form; it is preferred that the antibody described above is contained in about 5 to about 100 mg especially in the form of injection, and in 10 to 250 mg for the other forms.

The dose of the aforesaid prophylactic/therapeutic agent or regulator comprising the antibody of the present invention may vary depending upon subject to be administered, target disease, conditions, route of administration, etc. For example, when used for the purpose of treating/preventing, e.g., breast cancer in an adult, it is advantageous to administer the antibody of the present invention intravenously in a dose of about 0.01 to about 20 mg/kg body weight, preferably about 0.1 to about 10 mg/kg body weight and more preferably about 0.1 to about 5 mg/kg body weight, about 1 to 5 times/day, preferably about 1 to 3 times/day. In other parenteral and oral administration, the agent can be administered in a dose corresponding to the dose given above. When the condition is especially severe, the dose may be increased according to the condition.

The antibody of the present invention may be administered as it stands or in the form of an appropriate composition. The composition used for the administration may contain a pharmacologically acceptable carrier with the aforesaid antibody or its salts, a diluent or excipient. Such a composition is provided in the form of pharmaceutical preparations suitable for oral or parenteral administration (e.g., intravascular injection, subcutaneous injection, etc.). Each composition described above may further contain other active ingredients. Furthermore, the antibody of the present invention may be used in combination with other drugs, for example, alkylating agents (e.g., cyclophosphamide, ifosfamide, etc.), metabolic antagonists (e.g., methotrexate, 5-fluorouracil, etc.), anti-tumor antibiotics (e.g., mitomycin, adriamycin, etc.), plant-derived anti-tumor agents (e.g., vincristine, vindesine, Taxol, etc.), cisplatin, carboplatin, etoposide, irinotecan, etc. The antibody of the present invention and the drugs described above may be administered simultaneously or at staggered times to the patient.

The method of treatment described herein, particularly for cancers, may also be carried out with administration of other antibodies or chemotherapeutic agents. For example, an antibody against EGFR, such as ERBITUX® (cetuximab), may also be administered, particularly when treating colon cancer. ERBITUX® has also been shown to be effective for treatment of psoriasis. Other antibodies for combination use include Herceptin® (trastuzumab) particularly when treating breast cancer, AVASTIN® particularly when treating colon cancer and SGN-15 particularly when treating non-small cell lung cancer. The administration of the antibody of the present invention with other antibodies/chemotherapeutic agents may occur simultaneously, or separately, via the same or different route.

The chemotherapeutic agent/other antibody regimens utilized include any regimen believed to be optimally suitable for the treatment of the patient's condition. Different malignancies can require use of specific anti-tumor antibodies and specific chemotherapeutic agents, which will be determined on a patient to patient basis. In a preferred embodiment of the invention, chemotherapy is administered concurrently with or, more preferably, subsequent to antibody therapy. It should be emphasized, however, that the present invention is not limited to any particular method or route of administration.

The preponderance of evidence shows that AR47A6.4.2 mediates anti-cancer effects and prolongs survival through ligation of epitopes present on TROP-2. It has previously been shown (as disclosed in Ser. No. 11/709,676) that AR47A6.4.2 antibodies can be used to immunoprecipitate the cognate antigen from expressing cells such as MDA-MB-231 cells. Further it could be shown that AR47A6.4.2, chimeric AR47A6.4.2 ((ch)AR47A6.4.2) or humanized variants, (hu)AR47A6.4.2 can be used in the detection of cells and/or tissues which express a TROP-2 antigenic moiety which specifically binds thereto, utilizing techniques illustrated by, but not limited to FACS, cell ELISA or IHC.

As with the AR47A6.4.2 antibody, other anti-TROP-2 antibodies could be used to immunoprecipitate and isolate other forms of the TROP-2 antigen, and the antigen can also be used to inhibit the binding of those antibodies to the cells or tissues that express the antigen using the same types of assays.

### SEQ ID NOs

| | |
|---|---|
| SEQ | |
| ID NO: | Sequence |

| | | |
|---|---|---|
| Heavy | 1 | NYGMN |
| CDR1 | | |
| Heavy | 2 | WINTKTGEPTYAEEFKG |
| CDR2 | | |
| Heavy | 3 | GGYGSSYWYFDV |
| CDR3 | | |
| Light | 4 | KASQDVSIAVA |
| CDR1 | | |
| Light | 5 | SASYRYT |
| CDR2 | | |
| Light | 6 | QQHYITPLT |
| CDR3 | | |
| HV2 | 7 | Q I Q L V Q S G H E V K K P G A S V |
| | | K V S C K A S G Y T F T N Y G M N W |
| | | V R Q A P G Q G L E W M G W I N T K |
| | | T G E P T Y A E E F K G R F V F S L E |
| | | T S A S T A Y L Q I S S L K A E D T A |
| | | M Y F C G R G G Y G S S Y W Y F D V |
| | | W G Q G T T V T V S S |
| KV3 | 8 | D I Q M T Q S P S S L S A S V G D R V |
| | | T I T C K A S Q D V S I A V A W Y Q |
| | | Q K P G K A P K V L I Y S A S Y R Y |
| | | T G V P D R F S G S G S G T D F T F T |
| | | I S S L Q P E D I A V Y Y C Q Q H Y I |
| | | T P L T F G G G T K V E I K |
| KV4 | 9 | D I Q M T Q S P S S L S A S V G D R V |
| | | T I T C K A S Q D V S I A V A W Y Q |
| | | Q K P G K A P K V L I Y S A S Y R Y |
| | | T G V P S R F S G S G S G T D F T F T |
| | | I S S L Q P E D I A V Y Y C Q Q H Y I |
| | | T P L T F G G G T K V E I K |
| HV3 | 10 | Q I Q L V Q S G H E V K K P G A S V |
| | | K V S C K A S G Y T F T N Y G M N W |
| | | V R Q A P G Q G L E W M G W I N T K |
| | | T G E P T Y A E E F K G R F V F S L E |
| | | T S A S T A Y L Q I S S L K A E D M A |
| | | M Y F C G R G G Y G S S Y W Y F D V |
| | | W G Q G T T V T V S S |

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of reduction of a human pancreatic, breast, prostate, ovarian or colon tumor in a mammal, wherein said human pancreatic, breast, prostate, ovarian or colon tumor expresses at least one epitope of an antigen which specifically binds to the isolated monoclonal antibody produced by the hybridoma cell line deposited with the IDAC as accession number 141205-05 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's pancreatic, breast, prostate, ovarian or colon tumor burden.

2. The method of claim 1 wherein said isolated monoclonal antibody is conjugated to a cytotoxic moiety.

3. The method of claim 2 wherein said cytotoxic moiety is a radioactive isotope.

4. The method of claim 1 wherein said isolated monoclonal antibody or CDMAB thereof activates complement.

5. The method of claim 1 wherein said isolated monoclonal antibody or CDMAB thereof mediates antibody dependent cellular cytotoxicity.

6. The method of claim 1 wherein said isolated monoclonal antibody is a humanized antibody of the isolated monoclonal antibody produced by the hybridoma deposited with the IDAC as accession number 141205-05 or an antigen binding fragment produced from said humanized antibody.

7. The method of claim 1 wherein said isolated monoclonal antibody is a chimeric antibody of the isolated monoclonal antibody produced by the hybridoma deposited with the IDAC as accession number 141205-05 or an antigen binding fragment produced from said chimeric antibody.

8. A method of reduction of a human pancreatic, breast, prostate, ovarian or colon tumor susceptible to antibody induced cellular cytotoxicity in a mammal, wherein said human pancreatic, breast, prostate, ovarian or colon tumor expresses at least one epitope of an antigen which specifically binds to the isolated monoclonal antibody produced by the hybridoma cell line deposited with the IDAC as accession number 141205-05 or a CDMAB thereof, which CDMAB is characterized by an ability to competitively inhibit binding of said isolated monoclonal antibody to its target antigen, comprising administering to said mammal said monoclonal antibody or said CDMAB thereof in an amount effective to result in a reduction of said mammal's pancreatic, breast, prostate, ovarian or colon tumor burden.

9. The method of claim 8 wherein said isolated monoclonal antibody is conjugated to a cytotoxic moiety.

10. The method of claim 9 wherein said cytotoxic moiety is a radioactive isotope.

11. The method of claim 8 wherein said isolated monoclonal antibody or CDMAB thereof activates complement.

12. The method of claim 8 wherein said isolated monoclonal antibody or CDMAB thereof mediates antibody dependent cellular cytotoxicity.

13. The method of claim 8 wherein said isolated monoclonal antibody is a humanized antibody of the isolated monoclonal antibody produced by the hybridoma deposited with the IDAC as accession number 141205-05 or an antigen binding fragment produced from said humanized antibody.

14. The method of claim 8 wherein said isolated monoclonal antibody is a chimeric antibody of the isolated monoclonal antibody produced by the hybridoma deposited with the IDAC as accession number 141205-05 or an antigen binding fragment produced from said chimeric antibody.

15. A method of reduction of a human pancreatic, breast, prostate, ovarian or colon tumor in a mammal, wherein said human pancreatic, breast, prostate, ovarian or colon tumor expresses at least one epitope of an antigen which specifically binds to the isolated monoclonal antibody produced by the hybridoma deposited with the IDAC as accession number 141205-05 or a CDMAB thereof, which CDMAB is characterized by an ability to competitively inhibit binding of said isolated monoclonal antibody to its target antigen, comprising administering to said mammal said monoclonal antibody or CDMAB thereof in conjunction with at least one chemotherapeutic agent in an amount effective to result in a reduction of said mammal's pancreatic, breast, prostate, ovarian or colon tumor burden.

16. The method of claim 15 wherein said isolated monoclonal antibody is conjugated to a cytotoxic moiety.

17. The method of claim 16 wherein said cytotoxic moiety is a radioactive isotope.

18. The method of claim 15 wherein said isolated monoclonal antibody or CDMAB thereof activates complement.

19. The method of claim 15 wherein said isolated monoclonal antibody or CDMAB thereof mediates antibody dependent cellular cytotoxicity.

20. The method of claim 15 wherein said isolated monoclonal antibody is a humanized antibody of the isolated monoclonal antibody produced by the hybridoma deposited with the IDAC as accession number 141205-05 or an antigen binding fragment produced from said humanized antibody.

21. The method of claim 15 wherein said isolated monoclonal antibody is a chimeric antibody of the isolated monoclonal antibody produced by the hybridoma deposited with the IDAC as accession number 141205-05 or an antigen binding fragment produced from said chimeric antibody.

22. Use of monoclonal antibodies for reduction of human breast, pancreatic, ovarian, prostate or colon tumor burden, wherein said human breast, pancreatic, ovarian, prostate or colon tumor expresses at least one epitope of an antigen which specifically binds to the isolated monoclonal antibody produced by the hybridoma deposited with the IDAC as accession number 141205-05 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's human breast, pancreatic, ovarian, prostate or colon tumor burden.

23. The method of claim 22 wherein said isolated monoclonal antibody is conjugated to a cytotoxic moiety.

24. The method of claim 23 wherein said cytotoxic moiety is a radioactive isotope.

25. The method of claim 22 wherein said isolated monoclonal antibody or CDMAB thereof activates complement.

26. The method of claim 22 wherein said isolated monoclonal antibody or CDMAB thereof mediates antibody dependent cellular cytotoxicity.

27. The method of claim 22 wherein said isolated monoclonal antibody is a humanized antibody of the isolated monoclonal antibody produced by the hybridoma deposited with the IDAC as accession number 141205-05.

28. The method of claim 22 wherein said isolated monoclonal antibody is a chimeric antibody of the isolated monoclonal antibody produced by the hybridoma deposited with the IDAC as accession number 141205-05.

29. Use of monoclonal antibodies for reduction of human breast, pancreatic, ovarian, prostate or colon tumor burden, wherein said human breast, pancreatic, ovarian, prostate or colon tumor expresses at least one epitope of an antigen which specifically binds to the isolated monoclonal antibody produced by the hybridoma deposited with the IDAC as accession number 141205-05 or a CDMAB thereof, which CDMAB is characterized by an ability to competitively inhibit binding of said isolated monoclonal antibody to its target antigen, comprising administering to said mammal said monoclonal antibody or CDMAB thereof; in conjunction with at least one chemotherapeutic agent in an amount effective to result in a reduction of said mammal's human breast, pancreatic, ovarian, prostate or colon tumor burden.

30. The method of claim 29 wherein said isolated monoclonal antibody is conjugated to a cytotoxic moiety.

31. The method of claim 30 wherein said cytotoxic moiety is a radioactive isotope.

32. The method of claim 29 wherein said isolated monoclonal antibody or CDMAB thereof activates complement.

33. The method of claim 29 wherein said isolated monoclonal antibody or CDMAB thereof mediates antibody dependent cellular cytotoxicity.

34. The method of claim 29 wherein said isolated monoclonal antibody is a humanized antibody of the isolated monoclonal antibody produced by the hybridoma deposited with the IDAC as accession number 141205-05.

35. The method of claim 29 wherein said isolated monoclonal antibody is a chimeric antibody of the isolated monoclonal antibody produced by the hybridoma deposited with the IDAC as accession number 141205-05.

36. A process for reduction of a human breast, pancreatic, ovarian, prostate or colon tumor which expresses at least one epitope of human TROP-2 antigen which is specifically bound by the isolated monoclonal antibody produced by hybridoma cell line AR47A6.4.2 having IDAC Accession No. 141205-05, comprising:
administering to an individual suffering from said human tumor, at least one isolated monoclonal antibody or CDMAB thereof that binds the same epitope or epitopes as those bound by the isolated monoclonal antibody produced by the hybridoma cell line AR47A6.4.2 having IDAC Accession No. 141205-05;
wherein binding of said epitope or epitopes results in a reduction of breast, pancreatic, ovarian, prostate or colon tumor burden.

37. A process for reduction of a human breast, pancreatic, ovarian, prostate or colon tumor which expresses at least one epitope of human TROP-2 antigen which is specifically bound by the isolated monoclonal antibody produced by hybridoma cell line AR47A6.4.2 having IDAC Accession No. 141205-05, comprising:
administering to an individual suffering from said human tumor, at least one isolated monoclonal antibody or CDMAB thereof, that binds the same epitope or epitopes as those bound by the isolated monoclonal antibody produced by the hybridoma cell line AR47A6.4.2 having IDAC Accession No. 141205-05; in conjunction with at least one chemotherapeutic agent;
wherein said administration results in a reduction of tumor burden.

38. A binding assay to determine a presence of cancerous cells in a tissue sample selected from a human tumor, which is specifically bound by the isolated monoclonal antibody produced by hybridoma cell line AR47A6.4.2 having IDAC Accession No. 141205-05, the humanized antibody of the isolated monoclonal antibody produced by the hybridoma deposited with the IDAC as accession number 141205-05 or the chimeric antibody of the isolated monoclonal antibody produced by the hybridoma deposited with the IDAC as accession number 141205-05, comprising:
providing a tissue sample from said human tumor;
providing at least one of said isolated monoclonal antibody, said humanized antibody, said chimeric antibody or CDMAB thereof that recognizes the same epitope or epitopes as those recognized by the isolated monoclonal antibody produced by a hybridoma cell line AR47A6.4.2 having IDAC Accession No. 141205-05;
contacting at least one said provided antibodies or CDMAB thereof with said tissue sample; and determining binding of said at least one provided antibody or CDMAB thereof with said tissue sample;
whereby the presence of said cancerous cells in said tissue sample is indicated.

39. A binding assay to determine the presence of cells which express TROP-2 which is specifically recognized by the isolated monoclonal antibody produced by the hybridoma cell line AR47A6.4.2 having IDAC Accession No. 141205-05, the humanized antibody of the isolated monoclonal antibody produced by the hybridoma deposited with the IDAC as accession number 141205-05 or the chimeric antibody of the isolated monoclonal antibody produced by the hybridoma deposited with the IDAC as accession number 141205-05, comprising:
providing a cell sample;
providing the isolated monoclonal antibody produced by the hybridoma cell line AR47A6.4.2 having IDAC Accession No. 141205-05, said humanized antibody, said chimeric antibody or CDMAB thereof;
contacting said isolated monoclonal antibody or said antigen binding fragment with said cell sample; and
determining binding of said isolated monoclonal antibody or CDMAB thereof with said cell sample;
whereby the presence of cells which express an antigen of TROP-2 which is specifically bound by said isolated monoclonal antibody or said CDMBA thereof is determined.

40. A method for inducing complement dependent cytotoxicity of cancerous cells, which express at least one epitope of TROP-2 on the cell's surface, which at least one epitope, when bound by the isolated monoclonal antibody produced by the hybridoma deposited with the IDAC as 141205-05 or an antigen binding fragment produced from said isolated monoclonal antibody results in cell cytotoxicity, comprising:
providing the isolated monoclonal antibody produced by the hybridoma deposited with the IDAC as 141205-05 or an antigen binding fragment produced from said isolated monoclonal antibody, and
contacting said cancerous cells with said isolated monoclonal antibody or said antigen binding fragment;
whereby cytotoxicity occurs as a result of binding of said isolated monoclonal antibody or said antigen binding fragment with said at least one epitope of TROP-2.

41. The method of claim 40 wherein said isolated monoclonal antibody is conjugated to a cytotoxic moiety.

42. The method of claim 41 wherein said cytotoxic moiety is a radioactive isotope.

43. The method of claim 40 wherein said isolated monoclonal antibody activates complement.

44. The method of claim 40 wherein said isolated monoclonal antibody mediates cellular cytotoxicity.

45. The method of claim 40 wherein said monoclonal antibody is a humanized antibody of the isolated monoclonal antibody produced by the hybridoma deposited with the IDAC as 141205-05 or an antigen binding fragment produced from said humanized antibody.

46. The method of claim 40 wherein said monoclonal antibody is a chimeric antibody of the isolated monoclonal antibody produced by the hybridoma deposited with the IDAC as 141205-05 or an antigen binding fragment produced from said chimeric antibody.

47. A method for inducing complement dependent cytotoxicity of cancerous cells, which express at least one epitope of TROP-2 on the cell's surface, which at least one epitope, when bound by the isolated monoclonal antibody produced by the hybridoma deposited with the IDAC as 141205-05 or an antigen binding fragment produced from said isolated monoclonal antibody results in cell cytotoxicity, comprising:
providing an isolated monoclonal antibody which competitively inhibits binding of the isolated monoclonal antibody produced by the hybridoma deposited with the IDAC as 141205-05 or of an antigen binding fragment produced from said isolated monoclonal antibody, and which when bound by said at least one epitope of TROP-2, results in cell cytotoxicity; and
contacting said cancerous cells with said isolated monoclonal antibody or said antigen binding fragment;
whereby cytotoxicity occurs as a result of binding of said isolated monoclonal antibody or said antigen binding fragment with said at least one epitope of TROP-2.

48. A monoclonal antibody which specifically binds to the same epitope or epitopes as the isolated monoclonal antibody produced by the hybridoma deposited with the IDAC as accession number 141205-05.

49. An isolated monoclonal antibody or CDMAB thereof, which specifically binds to human TROP-2, in which the isolated monoclonal antibody or CDMAB thereof reacts with the same epitope or epitopes of human TROP-2 as the isolated monoclonal antibody produced by a hybridoma cell line AR47A6.4.2 having IDAC Accession No. 141205-05; said isolated monoclonal antibody or CDMAB thereof being characterized by an ability to competitively inhibit binding of said isolated monoclonal antibody to its target human TROP-2 antigen.

50. An isolated monoclonal antibody or CDMAB thereof that recognizes the same epitope or epitopes as those recognized by the isolated monoclonal antibody produced by the hybridoma cell line AR47A6.4.2 having IDAC Accession No. 141205-05; said monoclonal antibody or CDMAB thereof being characterized by an ability to competitively inhibit binding of said isolated monoclonal antibody to its target epitope or epitopes.

51. A humanized antibody that specifically binds the same epitope or epitopes of human TROP-2 as the isolated monoclonal antibody produced by the hybridoma cell line AR47A6.4.2 having IDAC Accession No. 141205-05, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6;
or a human TROP-2 binding fragment thereof.

52. A humanized antibody that specifically binds the same epitope or epitopes of human TROP-2 as the isolated monoclonal antibody produced by the hybridoma cell line AR47A6.4.2 having IDAC Accession No. 141205-05, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6; and variable domain framework regions from the heavy and light chains of a human antibody or human antibody consensus framework;
or a human TROP-2 binding fragment thereof.

53. A humanized antibody that specifically binds human TROP-2, wherein said monoclonal antibody comprises a heavy chain variable region amino acid sequence of SEQ ID NO:7; and a light chain variable region amino acid sequence selected of SEQ ID NO:8;
or a human TROP-2 binding fragment thereof.

54. A humanized antibody that specifically binds human TROP-2, wherein said monoclonal antibody comprises a heavy chain variable region amino acid sequence of SEQ ID NO:7; and a light chain variable region amino acid sequence selected of SEQ ID NO:9;
or a human TROP-2 binding fragment thereof.

55. A humanized antibody that specifically binds human TROP-2, wherein said monoclonal antibody comprises a heavy chain variable region amino acid sequence of SEQ ID NO:10; and a light chain variable region amino acid sequence selected of SEQ ID NO:8;
or a human TROP-2 binding fragment thereof.

56. A humanized antibody that specifically binds human TROP-2, wherein said monoclonal antibody comprises a heavy chain variable region amino acid sequence of SEQ ID NO:10; and a light chain variable region amino acid sequence selected of SEQ ID NO:9;
or a human TROP-2 binding fragment thereof.

57. A composition effective for treating a human pancreatic, prostate, ovarian, breast or colon tumor comprising in combination:
an antibody or CDMAB of any one of claims 1, 2, 3, 6, 7, 8, 17, 49, 50, 54, 55, or 56;
a conjugate of said antibody or an antigen binding fragment thereof with a member selected from the group consisting of cytotoxic moieties, enzymes, radioactive compounds, cytokines, interferons, target or reporter moieties and hematogenous cells; and
a requisite amount of a pharmacologically acceptable carrier;
wherein said composition is effective for treating said human pancreatic, breast, prostate, ovarian or colon tumor.

58. A composition effective for treating a human pancreatic, breast, prostate, ovarian or colon tumor comprising in combination:
an antibody or CDMAB of any one of claims 1, 2, 3, 6, 7, 8, 17, 49, 50, 54, 55, or 56; and a requisite amount of a pharmacologically acceptable carrier;
wherein said composition is effective for treating said human pancreatic, breast, prostate, ovarian or colon tumor.

59. A composition effective for treating a human pancreatic, breast, prostate, ovarian or colon tumor comprising in combination:
a conjugate of an antibody, antigen binding fragment, or CDMAB of any one of claims 1, 2, 3, 6, 7, 8, 17, 49, 50, 54, 55, or 56; with a member selected from the group consisting of cytotoxic moieties, enzymes, radioactive compounds, cytokines, interferons, target or reporter moieties and hematogenous cells; and
a requisite amount of a pharmacologically acceptable carrier;
wherein said composition is effective for treating said human pancreatic, breast, prostate, ovarian or colon tumor.

60. An assay kit for detecting the presence of a human cancerous tumor, wherein said human cancerous tumor expresses at least one epitope of an antigen which specifically binds to the isolated monoclonal antibody produced by the hybridoma deposited with the IDAC as accession number 141205-05 or a CDMAB thereof, which CDMAB is characterized by an ability to competitively inhibit binding of said isolated monoclonal antibody to its target antigen, the kit comprising the isolated monoclonal antibody produced by the hybridoma deposited with the IDAC as accession number 141205-05 or a CDMAB thereof, and means for detecting whether the monoclonal antibody, or a CDMAB thereof, is bound to a polypeptide whose presence, at a particular cut-off level, is diagnostic of said presence of said human cancerous tumor.
